SEQUENCE LISTING (17 sequences)

SEQ ID NO: 1 - DNA, 426 bp, Staphylococcus aureus
atgaaactaaaatcatttgttactgccactttagcattgggattattatcaacggtcggagctgcattaccgagtcacgaagcatctgcagatagtaataacggctataaagaaatgactgtggatggttatcacactgttccttacacaatttcagtagatggtattactgcattacatcgaacttactttatcttcccagaaaataaaaatgttctttatcaagaaattgacagtaaagtaaaaaatgaattagcttctcaacgtggtgttacaacagaaaaaattaataatgcccaaacagcaacttatacgcttactttgaatgatggtaataaaaaagtagtgaatctaaagaaaaatgacgacgctaaaaattcaattgatccaagtacaatcaaacagatacaaattgtagttaaataa

SEQ ID NO: 2 - protein, 141 aa, Staphylococcus aureus
MKLKSFVTATLALGLLSTVGAALPSHEASADSNNGYKEMTVDGYHTVPYTISVDGITALHRTYFIFPENKNVLYQEIDSKVKNELASQRGVTTEKINNAQTATYTLTLNDGNKKVVNLKKNDDAKNSIDPSTIKQIQIVVK

SEQ ID NO: 3 - DNA, 348 bp, Staphylococcus aureus
cacgaggcctctgccgatagcaacaacggctacaaagaaatgaccgtggatggctaccacaccgtgccttacaccatctctgtggatggaatcaccgccctgcaccggacctacttcatcttccccgagaacaagaacgtgctgtaccaggaaatcgactctaaagtgaagaacgagctggcctcccagagaggcgtgacaaccgagaagattaacaacgcccagaccgccacctacaccctgaccctgaacgacggcaacaaaaaggtcgtgaatctgaagaagaacgacgacgccaagaacagcatcgaccccagcaccattaagcagatccagatcgtcgtgaag

SEQ ID NO: 4 - protein, 116 aa, Staphylococcus aureus
HEASADSNNGYKEMTVDGYHTVPYTISVDGITALHRTYFIFPENKNVLYQEIDSKVKNELASQRGVTTEKINNAQTATYTLTLNDGNKKVVNLKKNDDAKNSIDPSTIKQIQIVVK

SEQ ID NO: 5 - DNA, 435 bp, Staphylococcus aureus
atgaaattaaaatcatttataactgtaactttggcactgggcatgatcgcaacgactggcgctactgtggcaggtaatgaggtatctgcagcagaaaaggacaaactaccggcaactcaaaaagctaaagaaatgcaaaatgttccatatacaattgcagtagatggcattatggctttcaatcaatcttacttaaatttaccaaaagatagccaattatcatatttagatttaggaaataaagttaaagctttgttatatgatgaacgcggtgtaacacctgagaagattcgaaatgcaaaatctgccgtttacacgattacttggaaagatggtagtaaaaaagaagtggatcttaagaaagatagctacacagcaaacttgtttgattcaaattcaattaaacaaattgatattaatgtaaaaactaaataa

SEQ ID NO: 6 - protein, 144 aa, Staphylococcus aureus
MKLKSFITVTLALGMIATTGATVAGNEVSAAEKDKLPATQKAKEMQNVPYTIAVDGIMAFNQSYLNLPKDSQLSYLDLGNKVKALLYDERGVTPEKIRNAKSAVYTITWKDGSKKEVDLKKDSYTANLFDSNSIKQIDINVKTK

SEQ ID NO: 7 - DNA, 357 bp, Staphylococcus aureus
aacgaggtgtcagccgccgagaaggataagctgcccgccacccagaaagccaaagaaatgcagaacgtgccctacacaatcgccgtggacggcatcatggccttcaaccagagctacctgaacctgcccaaggacagccagctgagctatctggacctgggcaacaaagtgaaggccctgctgtacgacgagcggggcgtgacccctgagaagatcagaaacgccaagagcgccgtgtacaccatcacctggaaggacggcagcaagaaagaggtggacctgaagaaggacagctacaccgccaacctgttcgacagcaacagcatcaagcagatcgacatcaacgtgaaaacaaag

SEQ ID NO: 8 - protein, 119 aa, Staphylococcus aureus
NEVSAAEKDKLPATQKAKEMQNVPYTIAVDGIMAFNQSYLNLPKDSQLSYLDLGNKVKALLYDERGVTPEKIRNAKSAVYTITWKDGSKKEVDLKKDSYTANLFDSNSIKQIDINVKTK

SEQ ID NO: 9 - protein, 104 aa, Staphylococcus aureus
VPYTIAVNGTSTPILSKLKISNKQLISYKYLNDKVKSVLKSERGISDLDLKFAKQAKYTVYFKNGKKQVVNLKSDIFTPNLFSAKDIKKIDIDVKQYTKSKKNK

SEQ ID NO: 10 - DNA, 335 bp, Artificial Sequence
  Codon-optimised Eap MAP
gttccggagtgccctataccattgctgtgaacggcaccagcacccccatcctgagcaagctgaagatcagcaacaagcagctgatctcctacaagtacctgaacgacaaagtgaaaagcgtgctgaagtccgagagaggcatcagcgacctggatctgaagttcgccaagcaggccaagtacaccgtgtatttcaagaacgggaagaaacaggtcgtgaacctgaaaagcgacatcttcacccctaatctgttcagcgccaaggacatcaagaaaattgatatcgacgtgaagcagtacaccaagagcaagaagaacaagggatccgggcccggg

SEQ ID NO: 11 - protein, 584 aa, Staphylococcus aureus
MKFKSLITTTLALGVLASTGANFNNNEASAAAKPLDKSSSSLHHGYSKVHVPYAITVNGTSQNILSSLTFNKNQNISYKDLEDRVKSVLKSDRGISDIDLRLSKQAKYTVYFKNGTKKVIDLKVGIYTADLINTSEIKAININVDTKKQVEDKKKDKANYQVPYTITVNGTSQNILSNLTFNKNQNISYKDLEDKVKSVLESNRGITDVDLRLSKQAKYTVNFKNGTKKVIDLKSGIYTANLINSSDIKSININVDTKKHIENKAKRNYQVPYSINLNGTSTNILSNLSFSNKPWTNYKNLTSQIKSVLKHDRGISEQDLKYAKKAYYTVYFKNGGKRILQLNSKNYTANLVHAKDVKRIEITVKTGTKAKADRYVPYTIAVNGTSTPILSDLKFTGDPRVGYKDISKKVKSVLKHDRGIGERELKYAKKATYAVHFKNGTKKVININSNISQLNLLYVQDIKKIDIDVKTGTKAKADSYVPYTIAVNGTSTPILSKLKISNKQLISYKYLNDKVKSVLKSERGISDLDLKFAKQAKYTVYFRNGKKQVVNLKSDIFTPNLFSAKDIKKIGIDVKQYTKSKKNK

SEQ ID NO: 12 - DNA, 2085 bp, Staphylococcus aureus
aattatcataaaaaaggagtgataatttatgaaatttaagtcattgattacaacaactttagcattaggtgttttagcatcaacaggtgcaaactttaataataatgaagcgtctgccgcagctaagccattagataaatcatcaagttcgttacaccatggatattctaaagtccatgttccatatgcaatcactgtgaacggtacaagccaaaatattttatcaagcttaacatttaataagaatcaaaatattagttataaagatttagaggatagagttaaatcagttttaaaatcagacagaggtattagtgatatagatttaagactatcgaagcaagcgaaatatactgtttactttaaaaatggaacaaagaaagttatcgatttgaaagtaggtatttacacagctgatttaattaatacaagtgaaattaaagctattaatattaacgtagatactaaaaagcaagttgaagataaaaagaaagataaagcaaattaccaagttccatacacaatcactgtgaacggtacaagccaaaatattttatcaaacttaacatttaataagaatcaaaatattagttacaaagatttagaggataaagttaaatcagttttagaatcaaatagaggtattaccgatgttgatttaagattatcgaagcaagcgaaatatacagttaattttaaaaatggaacgaagaaagttatcgatttgaaatcaggtatttacacagcgaatttaataaattcaagtgatattaaaagtatcaatattaacgtagatacaaaaaaacatatcgaaaataaagctaaaagaaactatcaagttccatattcaattaatttaaatggtacatctacaaacattttatcgaatctttcattttcaaataaaccttggacaaattacaaaaatttaactagtcaaataaaatcagtactgaagcatgatagaggtattagtgaacaagatttaaaatatgctaagaaagcttattatactgtttattttaaaaatggtggtaaaagaatcttacagttgaattcaaaaaattacacagcaaacttagttcatgcgaaagatgttaagagaattgaaattactgttaaaacaggaactaaagcgaaagcagacagatatgtaccatacacaattgcagtaaatggcacatcaacaccaattttatcagatttaaaatttacaggtgacccacgtgtaggctacaaagatatctctaaaaaagttaaatcagtactgaagcatgatagaggtatcggggaacgtgaattaaaatatgcaaaaaaagctacttacgcagtacattttaaaaatggaacgaaaaaagtgattaacataaattcaaatattagccaactgaatctgctttatgttcaagatattaaaaagatagatattgatgttaaaacaggaactaaagcgaaagcggatagctatgtaccatatacaattgcagtaaatggcacatcaacaccaattttatcaaaacttaaaatttcgaataaacaattaattagttacaaatatttaaacgacaaagtgaaatctgtattaaaaagtgaaagaggcatcagtgatcttgacttaaaatttgcgaaacaagcaaaatatacagtatatttcagaaatggaaagaaacaagtagtgaatttaaaatcagacatctttacacctaatttatttagtgccaaagatattaaaaagattggtattgatgtaaaacaatacactaaatcaaaaaaaaataaataaattctaataatgtgaaattcccagtaacaataaataaatttgaaaacatagtttcaaatgaatttgtgttctataatgcaagcaaaattacaattaatgatttaagtataaaacttaaatcagcaatggcaaatgatcaagggataactaaacatgacataggacttgctgaacgcgcagtgtataaagtgtattttaaaaatggttcgtcaaaatatgtagacttaaaaactgagtataaagatgaaagagtatttaaagcaactgacattaaaaaagtagaaattgaaattaaattttaa

SEQ ID NO: 13 - DNA, 87 bp, Artificial Sequence
  TPA leader
atgaagagagggctctgctgtgtgctgctgctgtgtggagcagtcttcgtttcgcccagccaggaaatccatgcccgattcagaaga

SEQ ID NO: 14 - DNA, 225 bp, Artificial Sequence
  BGH polyA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 15 - DNA, 350 bp, Artificial Sequence
  Synthesised Eap used in viral vector
aagttctgtttcagggcccggttccgtacaccatcgcggttaacggtacctctaccccgatcctgtctaaactgaaaatctctaacaaacagctgatctcttacaaatacctgaacgacaaagttaaatctgttctgaaatctgaacgtggtatctctgacctggacctgaaattcgcgaaacaggcgaaatacaccgtttacttcaaaaacggtaaaaaacaggttgttaacctgaaatctgacatcttcaccccgaacctgttctctgcgaaagacatcaaaaaaatcgacatcgacgttaaacagtacaccaaatctaaaaaaaacaaataaagctttctagaccat

SEQ ID NO: 16 - DNA, 392 bp, Artificial Sequence
  Synthesised EapH1 used in viral vector
aagttctgtttcagggcccgccgtctcacgaagcgtctgcggactctaacaacggttacaaagaaatgaccgttgacggttaccacaccgttccgtacaccatctctgttgacggtatcaccgcgctgcaccgtacctacttcatcttcccggaaaacaaaaacgttctgtaccaggaaatcgactctaaagttaaaaacgaactggcgtctcagcgtggtgttaccaccgaaaaaatcaacaacgcgcagaccgcgacctacaccctgaccctgaacgacggtaacaaaaaagttgttaacctgaaaaaaaacgacgacgcgaaaaactctatcgacccgtctaccatcaaacagatccagatcgttgttaaataaagctttctagaccat

SEQ ID NO: 17 - DNA, 401 bp, Artificial Sequence
  Synthesised EapH2 used in viral vector
aagttctgtttcagggcccggcgggtaacgaagtttctgcggcggaaaaagacaaactgccggcgacccagaaagcgaaagaaatgcagaacgttccgtacaccatcgcggttgacggtatcatggcgttcaaccagtcttacctgaacctgccgaaagactctcagctgtcttacctggacctgggtaacaaagttaaagcgctgctgtacgacgaacgtggtgttaccccggaaaaaatccgtaacgcgaaatctgcggtttacaccatcacctggaaagacggttctaaaaaagaagttgacctgaaaaaagactcttacaccgcgaacctgttcgactctaactctatcaaacagatcgacatcaacgttaaaaccaaataaagctttctagaccat

(19) United States
(12) Patent Application Publication
(10) Pub. No.: US 2019/0276502 A1
(43) Pub. Date: Sep. 12, 2019

(54) FUSION POLYPEPTIDES DERIVED FROM STAPHYLOCOCCUS AUREUS ANTIGENS

(71) Applicant: Oxford University Innovation Limited, Oxford (GB)

(72) Inventors: David WYLLIE, Oxfordshire (GB); Elizabeth ALLEN, Oxfordshire (GB)

(21) Appl. No.: 16/347,213

(22) PCT Filed: Nov. 2, 2017
(86) PCT No.: PCT/GB2017/053301

§ 371 Date: May 3, 2019

(30) Foreign Application Priority Data
Nov. 3, 2016 (GB) 1618541.5

(51) Int. Cl. C07K 14/31; C12N 7/00; C07K 16/12; A61K 39/085; A61P 31/04; A61P 13/12

(52) U.S. Cl. CPC C07K 14/31; C12N 7/00; C07K 16/1271; A61K 39/085; A61K 38/00; A61P 13/12; C07K 2319/00; C07K 2317/76; A61P 31/04

(57) ABSTRACT

The present invention relates to fusion polypeptides comprising polypeptides derived from Staphylococcus aureus antigens, as well as vectors constructs comprising nucleic acid molecules encoding the fusion polypeptides. More particularly, the fusion polypeptides comprise: (i) a first polypeptide, wherein the first polypeptide is an EapH1 polypeptide, or a derivative or variant thereof; and (ii) a second polypeptide, wherein the second polypeptide is an EapH2 polypeptide, or a derivative or variant thereof. The invention also relates to the use of these fusion polypeptides and vectors, inter alia, as immunogenic compositions, particularly as vaccine compositions.

The present invention relates to fusion polypeptides comprising polypeptides derived from Staphylococcus aureus antigens, as well as vectors comprising nucleic acid molecules encoding the fusion polypeptides. More particularly, the fusion polypeptides comprise: (i) a first polypeptide, wherein the first polypeptide is an EapH1 polypeptide, or a derivative or variant thereof; and (ii) a second polypeptide, wherein the second polypeptide is an EapH2 polypeptide, or a derivative or variant thereof.

The invention also relates to the use of these fusion polypeptides and vectors, inter alia, as immunogenic compositions, particularly as vaccine compositions.

Staphylococcus aureus is a common bacterium of human skin and nares which can turn into a versatile pathogen causing a plethora of diseases ranging from mild skin infections to life threatening endocarditis, pneumonia and sepsis [1]. S. aureus hospital and community acquired infections are a major health concern and economic burden, which is aggravated by the increasing incidence of antibiotic resistance and the rapidly decreasing rate of discovery for new therapeutics [2]. There is currently no licenced vaccine against S. aureus and two major Phase III clinical trials have been unsuccessful [3]. S. aureus carriage, a state in which persistent asymptomatic isolation of S. aureus can be observed, is present in 20-30% of human populations [4]. In the absence of a vaccine, prophylactic antibiotic therapy, and decolonisation to reduce the human bioburden of colonising S. aureus, have become the cornerstone of the prevention of S. aureus infections clinically [5-10].

A cardinal feature of severe S. aureus infection is the formation of deep abscesses [11], a process in which both extracellular and intracellular S. aureus participate [12]. Interrupting the process of bacterial invasion is one route to reducing S. aureus disease, independent of effects of colonisation.

Staphylococcus aureus transcription is highly dynamic, with multiple virulence and immune evasion proteins produced in response to external stimuli, including neutrophils [13]. It appears that part of this dynamic response includes production of a range of immune subversion proteins neutralising critical aspects of the innate and adaptive immune attack upon it [14]. Neutralising S. aureus subversion proteins has been proposed as one route to an effective S. aureus vaccine [15]. However, given the large number of proteins apparently involved, and the pleotropic mechanisms deployed by S. aureus to subvert the mammalian immune system the optimal method of generating protective responses against them [16, 17], remains unclear.

S. aureus extracellular adhesion protein (Eap) is a virulence factor which is conserved in 97.5% of clinical S. aureus isolates [42, 43]. It is deposited within the pseudocapsule that encloses staphylococcal abscess communities [22]. Eap is involved in bacterial agglutination, tissue adherence and inhibition of neutrophil recruitment [23, 24]. It also blocks classical and lectin complement pathways at the level of C3 proconvertase formation which impairs phagocytosis and killing by neutrophils [25]. Eap has been implicated in bacterial persistence within host tissues in the renal abscess model of infection [22]. Immunization of mice with recombinant Eap resulted in a reduction of bacterial load and the number of abscesses formed during infection [22].

The Eap protein consists of four to six repetitive MHC class II analogue protein (MAP) domains connected with short linkers that are susceptible to proteolysis [21]. S. aureus strains also produce two structural Eap homologues: EapH1 and EapH2. These additional proteins are highly conserved, present in nearly all S. aureus strains studied [43]. They consist of a single MAP domain preceded by a stretch of 10-20 amino acids and an N-terminal signal sequence [44]. One of them is strongly upregulated on intracellular growth, according to microarray data [18]. All three Eap family proteins have been shown to interact with, and inhibit to neutrophil serine proteases, thus protecting other staphylococcal virulence factors against proteolytic degradation [19, 20]. Deletion of all three proteins, but not individual members, attenuates virulence [20].

Replication-incompetent viral vectors, such as adenovirus and MVA, have promise in eliciting a combination of B and T cell immunity against antigens they express [45]. They have been extensively studied in the prevention of malaria, tuberculosis, and a range of other pathogens [46] which can be controlled by a combination of T cell and antibody mediated immunity. Viral vector vaccination regimes may also relevant for Staphylococcus aureus, for which both T cell mediated and antibody based protection [16, 47] has been demonstrated in various animal models.

There is therefore a need for new immunogenic compositions that demonstrate improved immunogenicity when used in the prevention and treatment of S. aureus infections, in particular in human subjects. In particular, there is a need for new immunogenic compositions that can produce an improved antigen-specific T cell response, as well as an improved antibody response.

The inventors have now found that fusion polypeptides based on amino acid sequences from the EapH1 and EapH2 polypeptides, optionally in combination with other amino acid sequences, can form a part of an effective anti-S. aureus vaccine. In particular, it is shown herein that prime-boost vaccination with such fusion polypeptides resulted in a significant decrease in the number of abscesses formed in the murine renal abscess model of infection, a decrease in the bacterial burden in the murine renal abscess model of infection, and a decrease in S. aureus carriage in colonised mice, indicating the value of such fusion polypeptides and viral vectors as a vaccine. It is also shown herein that single doses of viral vectors expressing these antigens, administered intranasally, accelerate the loss of S. aureus carriage following exposure.

The present invention therefore addresses one or more of the above problems by providing fusion polypeptides and viral vectors, inter alia, for the prevention and/or treatment of S. aureus infections and carriage states.

The fusion polypeptides and viral vectors, inter alia, of the invention enable an immune response against S. aureus to be stimulated in an individual, and provide improved immunogenicity and efficacy.

In one embodiment, the invention provides a fusion polypeptide comprising:
(i) a first polypeptide, wherein the first polypeptide is an EapH1 polypeptide, or a derivative or variant thereof; and
(ii) a second polypeptide, wherein the second polypeptide is an EapH2 polypeptide, or a derivative or variant thereof.

The fusion polypeptide may additionally comprise:
(iii) a third polypeptide, wherein the third polypeptide comprises at least one Eap MAP domain,
   or a derivative or variant thereof.

Preferably, the first polypeptide:
(a) is a polypeptide which has an amino acid sequence having at least 80%, 85%, 90%, 93%, 95% or 99% identity to SEQ ID NO: 2 or SEQ ID NO: 4, or
(b) is a fragment of polypeptide (a), wherein the first polypeptide has the ability to induce antibodies which cross-react with a Staphylococcus EapH1 polypeptide.

Preferably, the first polypeptide is a polypeptide which has an amino acid sequence having at least 93% amino acid sequence identity to SEQ ID NO: 4.

Preferably, the second polypeptide:
(c) is a polypeptide which has an amino acid sequence having at least 80%, 85%, 90%, 95%, 97% or 99% identity to SEQ ID NO: 6 or SEQ ID NO: 8, or
(d) is a fragment of polypeptide (c),
   wherein the second polypeptide has the ability to induce antibodies which cross-react with a Staphylococcus EapH2 polypeptide.

Preferably, the second polypeptide is a polypeptide which has an amino acid sequence having at least 97% amino acid sequence identity to SEQ ID NO: 8.

Preferably, the third polypeptide:
(e) is a polypeptide which has an amino acid sequence having at least 80%, 85%, 90% or 95% identity to SEQ ID NO: 9, or
(f) is a fragment of polypeptide (e),
   wherein the third polypeptide has the ability to induce antibodies which cross-react with a Staphylococcus Eap polypeptide.

In some embodiments, the invention provides a polypeptide comprising or consisting of the first polypeptide (e.g. in the absence of the second polypeptide), optionally fused to the third polypeptide. References herein to a “fusion polypeptide” encompass these polypeptides also.

In some other embodiments, the invention provides a polypeptide comprising or consisting of the second polypeptide (e.g. in the absence of the first polypeptide), optionally fused to the third polypeptide. References herein to a “fusion polypeptide” encompass these polypeptides also.

The fusion polypeptide generally comprises at least two parts, e.g. the first and second polypeptides. It may additionally comprise a third part, i.e. the third polypeptide, or more parts. These polypeptides (and any other elements) are joined contiguously or are joined by amino acid linkers.

As mentioned above, the extracellular adhesion protein (Eap) is a virulence factor produced by S. aureus. Two molecules with some homology to Eap are also produced by S. aureus: EapH1 and EapH2. These additional proteins are highly conserved and present in nearly all S. aureus strains studied [43]. Both have an N-terminal signal sequence [44].

The complete nucleotide sequence of the EapH1 polypeptide from S. aureus Newman is given in WP_001549607.1 and also herein as SEQ ID NO: 1. The corresponding amino acid sequence is given as SEQ ID NO: 2. The EapH1 MAP domain amino acid sequence from S. aureus Newman is given in SEQ ID NO: 4. As used herein, the term “EapH1 polypeptide” includes polypeptides of SEQ ID NOs: 2 and 4.

The complete nucleotide sequence of the EapH2 polypeptide from S. aureus Newman is given in WP_000769689.1 and also herein as SEQ ID NO: 5. The corresponding amino acid sequence is given as SEQ ID NO: 6. The EapH2 MAP domain amino acid sequence from S. aureus Newman is given in SEQ ID NO: 8. As used herein, the term “EapH2 polypeptide” includes polypeptides of SEQ ID NOs: 6 and 8.

The amino acid sequence of the Eap polypeptide MAP domain from S. aureus Newman is given herein as SEQ ID NO: 9. Depending on the S. aureus strain, the mature Eap protein is approximately 50-70 kDa. It consists of four to six repetitive MAP domains connected with short linkers that are susceptible to proteolysis [21].

In some embodiments, the amino acid sequences of the EapH1 and EapH2 polypeptides correspond directly to wild-type (i.e. naturally-occurring) sequences.

Preferably, the EapH1 and/or EapH2 polypeptides are from S. aureus. Most preferably, the EapH1 and/or EapH2 polypeptides are from S. aureus Newman.

In some embodiments, the amino acid sequences of the EapH1 and EapH2 polypeptides may correspond to non-natural sequences, e.g. variants or derivatives of wild-type sequences.

As used herein, the term “derivative or variant” of a reference polypeptide refers to polypeptides having one or more amino acid changes compared to the amino acid sequence of the reference polypeptide. In particular, the EapH1 and EapH2 polypeptides may independently comprise one or more (e.g. 1-20 or 1-10) amino acid sequence modifications compared to wild-type sequences.

Such modifications include amino acid substitutions, additions and deletions, preferably conservative amino acid substitutions.

Conservative substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine.

Preferably, the modifications do not significantly affect the folding or activity of the polypeptide. They include small deletions, typically of 1 to about 30 amino acids (such as 1-10, or 1-5 amino acids); and small amino- or carboxyl-terminal extensions, such as an amino-terminal methionine residue, a small linker peptide of up to about 20-25 residues, or an affinity tag.

The polypeptides of the invention may also comprise non-naturally occurring amino acid residues. In this regard, in addition to the 20 standard amino acids, non-standard amino acids (such as 4-hydroxyproline, 6-N-methyl lysine, 2-aminoisobutyric acid, isovaline and a methyl serine) may be substituted for amino acid residues of the mycobacterial polypeptides of the present invention. A limited number of non-conservative amino acids, amino acids that are not encoded by the genetic code, and unnatural amino acids may be substituted for mycobacterial polypeptide amino acid residues. Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine.

Several methods are known in the art for incorporating non-naturally occurring amino acid residues into polypeptides. For example, an in vitro system can be employed wherein nonsense mutations are suppressed using chemically aminoacylated suppressor tRNAs.

Methods for synthesizing amino acids and aminoacylating tRNAs are known in the art. Transcription and translation of plasmids containing nonsense mutations can be carried out in a cell free system comprising an E. coli S30 extract and commercially available enzymes and other reagents. Peptides can be, for instance, purified by chromatography. In a second method, translation is carried out in Xenopus oocytes by microinjection of mutated mRNA and chemically aminoacylated suppressor tRNAs. Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. Naturally-occurring amino acid residues can be converted to non-naturally occurring species by in vitro chemical modification. Chemical modification can be combined with site-directed mutagenesis to further expand the range of substitutions.

Essential amino acids, such as those in the polypeptides of the present invention, can be identified according to procedures known in the art, such as site-directed mutagenesis or alanine-scanning mutagenesis. Sites of biological interaction can also be determined by physical analysis of structure, as determined by such techniques as nuclear magnetic resonance, crystallography, electron diffraction or photoaffinity labelling, in conjunction with mutation of putative contact site amino acids. The identities of essential amino acids can also be inferred from analysis of homologies with related family members of the polypeptide of interest.

A variant or derivative of a polypeptide of the invention may contain one or more analogues of an amino acid (e.g. an unnatural amino acid), or a substituted linkage, as compared with the sequence of the reference polypeptide. In a further embodiment, a polypeptide of interest may be a mimic of the reference polypeptide, which mimic reproduces at least one epitope of the reference polypeptide.

Variants and derivatives of the disclosed polynucleotide and polypeptide sequences of the invention can be generated through DNA shuffling. Briefly, variant DNAs may be generated by in vitro homologous recombination by random fragmentation of a parent DNA followed by reassembly using PCR, resulting in randomly introduced point mutations. This technique can be modified by using a family of parent DNAs, to introduce additional variability into the process. Selection or screening for the desired activity, followed by additional iterations of mutagenesis and assay provides for rapid “evolution” of sequences by selecting for desirable mutations while simultaneously selecting against detrimental changes.

Multiple amino acid substitutions can be made and tested using known methods of mutagenesis and screening. Methods are known for simultaneously randomizing two or more positions in a polypeptide, selecting for functional polypeptide, and then sequencing the mutagenized polypeptides to determine the spectrum of allowable substitutions at each position. Other methods that can be used include phage display.

Preferably, the first polypeptide is a polypeptide which has an amino acid sequence having at least 70% identity to SEQ ID NO: 2 or SEQ ID NO: 4.

Preferably, the second polypeptide is a polypeptide which has an amino acid sequence having at least 70% identity to SEQ ID NO: 6 or SEQ ID NO: 8.

Preferably, the third polypeptide is a polypeptide which has an amino acid sequence having at least 70% identity to SEQ ID NO: 9.

More preferably, the first polypeptide is a polypeptide which has an amino acid sequence having at least 75%, 80%, 85%, 90%, 93%, 95%, 96%, 97%, 98%, 99% or 100% amino acid sequence identity to SEQ ID NO: 2 or SEQ ID NO: 4.

More preferably, the second polypeptide is a polypeptide which has an amino acid sequence having at least 75%, 80%, 85%, 90%, 95%, 96%, 97%, 98%, 99% or 100% amino acid sequence identity to SEQ ID NO: 6 or SEQ ID NO: 8.

More preferably, the third polypeptide is a polypeptide which has an amino acid sequence having at least 75%, 80%, 85%, 90%, 95%, 96%, 97%, 98%, 99% or 100% amino acid sequence identity to SEQ ID NO: 9.

The first polypeptide is not an EapH2 polypeptide. The first polypeptide is not a polypeptide which has an amino acid sequence having at least 60% identity to SEQ ID NO: 6 or SEQ ID NO: 8.

The second polypeptide is not an EapH1 polypeptide. The second polypeptide is not a polypeptide which has an amino acid sequence having at least 60% identity to SEQ ID NO: 2 or SEQ ID NO: 4.

In some embodiments, the first polypeptide is a fragment of a polypeptide which has the above-mentioned amino acid sequence identities to SEQ ID NO: 2 or SEQ ID NO: 4. In some embodiments, the second polypeptide is a fragment of a polypeptide which has the above-mentioned amino acid sequence identities to SEQ ID NO: 6 or SEQ ID NO: 8. In some embodiments, the third polypeptide is a fragment of a polypeptide which has the above-mentioned amino acid sequence identities to SEQ ID NO: 9.

Preferably, the fragment is at least 50%, 60%, 70%, 80%, 90% or 95% of the length of the polypeptide which has the above-mentioned amino acid sequence identities to SEQ ID NOs: 2 or 4, or SEQ ID NOs: 6 or 8, or SEQ ID NO: 9, respectively.

The “fragment” generally comprises a series of consecutive amino acid residues from the sequence of said polypeptide. By way of example, a “fragment” of a polypeptide of interest may comprise (or consist of) at least 20 consecutive amino acid residues from the sequence of said polypeptide (e.g. at least 25, 30, 35, 40, 45, 50, 75, 80, 90 or 100, consecutive amino acid residues of said polypeptide). A fragment may include at least one epitope of the polypeptide of interest.

Routine deletion analyses of nucleic acid molecules can be performed to obtain functional fragments of a nucleic acid molecule that encodes a polypeptide of the invention. As an illustration, DNA molecules can be digested with Bal31 nuclease to obtain a series of nested deletions. These DNA fragments are then inserted into expression vectors in proper reading frame, and the expressed polypeptides are isolated and tested for the desired activity. An alternative to exonuclease digestion is to use oligonucleotide-directed mutagenesis to introduce deletions, or stop codons to specify production of a desired fragment. Alternatively, particular polynucleotide fragments can be synthesized using the polymerase chain reaction.

Mutagenesis methods as disclosed above can be combined with high-throughput screening methods to detect activity of cloned variant polypeptides. Mutagenized nucleic acid molecules that encode polypeptides of the invention, or fragments thereof, can be recovered from the host cells and rapidly sequenced using modern equipment.

These methods allow the rapid determination of the importance of individual amino acid residues in a polypeptide of interest, and can be applied to polypeptides of unknown structure.

Preferably, the first polypeptide has the ability to induce antibodies which cross-react with EapH1 and/or EapH2 polypeptides (e.g. of SEQ ID NOs: 2 or 4, or 6 or 8, respectively).

Preferably, the second polypeptide has the ability to induce antibodies which cross-react with EapH1 and/or EapH2 polypeptides (e.g. of SEQ ID NOs: 2 or 4, or 6 or 8, respectively).

Preferably, the third polypeptide has the ability to induce antibodies which cross-react with a Staphylococcus Eap polypeptide.

In some preferred embodiments, the EapH1 polypeptide and/or EapH2 polypeptide and/or Staphylococcus Eap polypeptide is a Staphylococcus aureus polypeptide.

In some particularly preferred embodiments, the Staphylococcus aureus EapH1 polypeptide has the amino acid sequence as given in SEQ ID NO: 2 or 4.

In some particularly preferred embodiments, the Staphylococcus aureus EapH2 polypeptide has the amino acid sequence as given in SEQ ID NO: 6 or 8.

In some particularly preferred embodiments, the Staphylococcus aureus Eap MAP domain has the amino acid sequence as given in SEQ ID NO: 9.

As used herein, the term “has the ability to induce antibodies” refers to the capability of the first, second and third polypeptides to induce antibodies against a Staphylococcus EapH1 or EapH2 polypeptide or Eap MAP domain polypeptide, respectively, in a subject when the polypeptides or viral vectors producing them are administered in a suitable manner into that subject. Preferably, the subject is a human subject. Methods of administering polypeptides in a manner which is suitable for inducing antibodies in subjects are well known in the art (as discussed further herein).

In some embodiments, the first and/or second and/or third polypeptides may share a common ability with their reference polypeptides to induce a T-cell response and/or a B-cell response. Immunological assays for measuring and quantifying T-cell responses and B-cell responses are well known in the art.

Hence in some embodiments, the invention provides a fusion polypeptide comprising:
(i) a first polypeptide, wherein the first polypeptide:
   (a) is a polypeptide which has an amino acid sequence having at least 90% identity to SEQ ID NO: 2 or SEQ ID NO: 4, or
   (b) is a fragment of polypeptide (a),
      wherein the first polypeptide has the ability to induce a T-cell response or a B-cell response against a Staphylococcus EapH1 polypeptide (e.g. one having SEQ ID NO: 2 or 4); and
(ii) a second polypeptide, wherein the second polypeptide:
   (c) is a polypeptide which has an amino acid sequence having at least 90% identity to SEQ ID NO: 6 or SEQ ID NO: 8, or
   (d) is a fragment of polypeptide (c),
      wherein the second polypeptide has the ability to induce a T-cell response or a B-cell response against a Staphylococcus EapH2 polypeptide (e.g. one having SEQ ID NO: 6 or 8); and optionally
(iii) a third polypeptide, wherein the third polypeptide:
   (e) is a polypeptide which has an amino acid sequence having at least 80% identity to SEQ ID NO: 9, or
   (f) is a fragment of polypeptide (e),
      wherein the third polypeptide has the ability to induce a T-cell response or a B-cell response against a Staphylococcus Eap polypeptide (e.g. one having SEQ ID NO: 9).

In some embodiments, the fusion polypeptide comprises:
(i) at least one EapH1 MAP domain or a derivative or variant thereof, and
(ii) at least one EapH2 MAP domain or a derivative or variant thereof, and optionally
(iii) at least one Eap MAP domain or a derivative or variant thereof.

In this context, the term “at least one” independently includes 1, 2, 3, 4, 5 or 6, preferably 1-4 or 1-2, and most preferably 1.

In some embodiments, the fusion polypeptide comprises less than 5, 4, 3 or 2 Eap MAP domains, or derivatives or variants thereof.

The first and second polypeptides may be present in either N- to C-terminal order.

In some embodiments, in addition to the first and second polypeptides, the fusion polypeptide may additionally comprise one or more other polypeptides.

One example of such another polypeptide is the Eap MAP domain, as discussed above.

Other examples of other polypeptides include the Staphylococcus BitC polypeptide and the Staphylococcus EsxA polypeptide. Preferably, the latter polypeptides are from S. aureus. Further details of the latter polypeptides may be obtained from WO2014/053861, the contents of which (including the specific BitC and EsxA sequences) are specifically incorporated herein by reference.

The other polypeptide may, for example, be a polypeptide antigen which is presented on a microbe, parasite or neoplasm, or a variant or derivative thereof.

The other polypeptide may, for example, be a viral, bacterial, protozoan, animal, mammalian or human polypeptide antigen, or a variant or derivative thereof.

In some embodiments, the antigen is from a disease-causing bacteria, disease-causing parasite or disease-causing virus, or a variant or derivative thereof. For example, the antigen may be from a malaria-causing parasite or an influenza-causing virus.

Preferably, the bacterial or parasite antigen is from or derived from Staphylococcus, pathogenic Neisseria, Mycobacteria, Escherichia or from Apicomplexa (e.g. Plasmodium) or helminths. More preferably, the bacterial or parasite antigen is from or derived from S. aureus, pathogenic Neisseria species, M. tuberculosis, E. coli or P. falciparum.

In other embodiments, the other polypeptide may, for example, be an polypeptide selected from the group consisting of ClfA, ClfB, FnBPA, FnBP, SdrC, SdrD, SdrE, SasA, SasB, SasC, SasD, SasX, SasF, SasG/AAp, MntC, IsdA, IsdB, IsdH, FhuD2, EsxA, EsxB, Spa, Coa, vWbp, Hla, HIgA, HIgB, HIgC, LukA, LukB, LukD, LukE, EpiP, Can, CsalA, Csa1B, Csa1C, Csa1D, CsA2A, Csa3A, Csa3B, CsA3C, Csa3D, Csa3E, Csa3G, Csa3H, Csa3I, Csa3J, Csa4A, Csa4B, Csa4c, scn, efb, efbc, or a variant or derivative thereof which maintains the immunogenic potential of the polypeptide.

In one embodiment, the other polypeptide is S. aureus BitC, a cell surface lipoprotein [19], with accession number NP_370379.

In another embodiment, the other polypeptide is the extracellular domain of the S. aureus Clumping factor B precursor [20] (ClfB, with accession YP_001333563).

In another embodiment, the other polypeptide is the S. aureus alpha toxin or a truncated form thereof (e.g. amino acids 1-75 or tHla75).

In another embodiment, the other polypeptide is the P. falciparum protein Pfs25.

The fusion polypeptide may comprise an N-terminal signal sequence. This may be used to mediate targeting of the fusion polypeptide to the endoplasmic reticulum (ER).

Signal sequences generally have a tripartite structure, consisting of a hydrophobic core region (h-region) flanked by an N- and C-region. The latter contains the signal peptidase (SPase) consensus cleavage site. Usually, signal sequences are cleaved off co-translationally; the resulting cleaved signal sequences are termed signal peptides.

Preferably, the fusion polypeptide additionally comprises a tPA (tissue plasminogen activator) signal sequence.

The amino acid sequence of one tPA signal sequence is given herein in SEQ ID NO: 13.

In other embodiments, the tPA signal sequence is an amino acid sequence having at least 80% sequence identity to SEQ ID NO: 13 (preferably at least 85%, 90%, 95%, 98%, or 99% sequence identity) and having the ability to target the fusion polypeptide to the endoplasmic reticulum.

In some embodiments, one or more elements of the fusion polypeptide are independently separated from one another by one or more linker peptides. For example, each linker peptide may comprise 1, 2, 3, 4, 5, 6, 7, 8, 9 or 10 amino acids.

When present, the linker amino acids should not significantly affect (i.e. significantly reduce) the ability of the fusion polypeptide to induce antibodies against S. aureus.

Preferably, no linker amino acids are used between the EapH1 and Eap H2 polypeptides.

In a further embodiment, the invention provides a nucleic acid molecule which codes for a fusion polypeptide of the invention.

The complete nucleotide sequence of the EapH1 polypeptide from S. aureus Newman is given in WP_001549607.1 and also herein as SEQ ID NO: 1.

The complete nucleotide sequence of the EapH2 polypeptide from S. aureus Newman is given in WP_000769689.1 and also herein as SEQ ID NO: 5.

In one embodiment, the invention provides a nucleic acid molecule comprising:
(i) SEQ ID NO: 1, or a variant thereof having at least 85%, 90% or 95% nucleotide sequence identity thereto; and
(ii) SEQ ID NO: 5, or a variant thereof having at least 85%, 90% or 95% nucleotide sequence identity thereto,
   wherein the nucleic acid molecule encodes a fusion polypeptide which is capable of inducing an immune response against a S. aureus EapH1 and/or EapH2 polypeptide in a subject.

As used herein, the terms “nucleic acid sequence”, “nucleic acid molecule” and “polynucleotide” are used interchangeably and do not imply any length restriction. These include DNA (including cDNA) and RNA sequences; and single- and double-stranded sequences.

The nucleic acid molecules of the present invention include isolated nucleic acid molecules that have been removed from their naturally-occurring environment, recombinant or cloned DNA isolates, and chemically-synthesized analogues or analogues which have been synthesized biologically by heterologous systems.

The nucleic acid molecules of the present invention may be prepared by any means known in the art. For example, large amounts of the polynucleotides may be produced by replication in a suitable host cell. The natural or synthetic DNA fragments coding for a desired fragment may be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The nucleic acid molecules of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

The original (e.g. wild-type) codons in a nucleic acid molecule may be optimized for expression in a desired cell line, for example, using an online tool such as that available at http://genomes.urv.es/OPTIMIZER/.

In one embodiment of the invention, therefore, the nucleic acid molecule is codon-optimized for expression in a host cell, preferably a human cell.

The invention also provides a vector or plasmid comprising a nucleic acid molecule of the invention. Preferably, the vector is an expression vector.

The vector and/or plasmid may comprise one or more regulatory sequences which are operably linked to the sequence which encodes the fusion polypeptide, e.g. one or more enhancer, promoter and/or transcriptional terminator sequences.

In some embodiments, the vector is viral vector, e.g. a poxvirus vector. In other embodiments, the vector is an adenoviral vector or a Modified Vaccinia Ankara (MVA) viral vector. Preferably, the vector is a non-replicating vector.

Non-replicating poxviruses and adenoviruses represent groups of viruses which may be used as vectors for the delivery of genetic material into a target cell. Viral vectors serve as antigen delivery vehicles and also have the power to activate the innate immune system through binding cell surface molecules that recognise viral elements. A recombinant viral vector can be produced that carries nucleic acid encoding a given antigen. The viral vector can then be used to deliver the nucleic acid to a target cell, where the encoded antigen is produced by the target cell's own molecular machinery. As “non-self”, the produced antigen generates an immune response in the target subject.

Without wishing to be bound by any one particular theory, the inventors believe that antigen delivery using the vectors of the invention stimulates, amongst other responses, a T-cell response in the subject. Thus, the inventors believe that one way in which the present invention provides for protection against S. aureus infection is by stimulating T-cell responses and the cell-mediated immunity system. In addition, humoral (antibody) based protection can also be achieved.

The vector of the invention may be a non-replicating poxvirus vector. As used herein, a non-replicating (or replication-deficient) viral vector is a viral vector which lacks the ability to productively replicate following infection of a target cell. Thus, a non-replicating viral vector cannot produce copies of itself following infection of a target cell. Non-replicating viral vectors may therefore advantageously have an improved safety profile as compared to replication-competent viral vectors.

In one embodiment, the non-replicating poxvirus vector is selected from a Modified Vaccinia virus Ankara (MVA) vector, a NYVAC vaccinia virus vector, a canarypox (ALVAC) vector, and a fowlpox (FPV) vector. MVA and NYVAC are both attenuated derivatives of vaccinia virus. Compared to vaccinia virus, MVA lacks approximately 26 of the approximately 200 open reading frames.

In one embodiment, the non-replicating poxvirus vector is an MVA vector.

The vector of the invention may be an adenovirus vector. In one embodiment, the adenovirus vector is a non-replicating adenovirus vector (wherein non-replicating is defined as above). Adenoviruses can be rendered non-replicating by deletion of the EI or both the EI and E3 gene regions. Alternatively, an adenovirus may be rendered non-replicating by alteration of the EI or of the EI and E3 gene regions such that said gene regions are rendered non-functional. For example, a non-replicating adenovirus may lack a functional EI region or may lack functional EI and E3 gene regions. In this way the adenoviruses are rendered replication-incompetent in most mammalian cell lines and do not replicate in immunised mammals. Most preferably, both EI and E3 gene region deletions are present in the adenovirus, thus allowing a greater size of transgene to be inserted. This is particularly important to allow larger antigens to be expressed, or when multiple antigens are to be expressed in a single vector, or when a large promoter sequence, such as the CMV promoter, is used. Deletion of the E3 as well as the EI region is particularly favoured for recombinant Ad5 vectors. Optionally, the E4 region can also be engineered.

In one embodiment, the adenovirus vector is selected from a human adenovirus vector, a simian adenovirus vector, a group B adenovirus vector, a group C adenovirus vector, a group E adenovirus vector, an adenovirus 6 vector, a PanAd3 vector, an adenovirus C3 vector, a ChAdY25 vector, an AdC68 vector, and an Ad5 vector.

The viral vector of the invention, as described above, can be used to deliver a single antigen to a target cell. Advantageously, the viral vector of the invention can also be used to deliver multiple (different) antigens to a target cell.

In one embodiment, the vector of the invention further comprises a nucleic acid sequence encoding an adjuvant (for example, a cholera toxin, an E. coli lethal toxin, or a flagellin).

The nucleic acid sequence encoding a vector (as described above) may be generated by the use of any technique for manipulating and generating recombinant nucleic acid known in the art. In one aspect, the invention provides a method of making a vector (as described above), comprising providing a nucleic acid, wherein the nucleic acid comprises a nucleic acid molecule encoding a vector of the invention; transfecting a host cell with the nucleic acid molecule; culturing the host cell under conditions suitable for the propagation of the vector; and obtaining the vector from the host cell.

As used herein, “transfecting” may mean any non-viral method of introducing nucleic acid molecules into a cell. The nucleic acid molecule may be any nucleic acid molecule suitable for transfecting a host cell. Thus, in one embodiment, the nucleic acid molecule is a plasmid. The host cell may be any cell in which a vector (i.e. a non-replicating poxvirus vector or an adenovirus vector, as described above) may be grown. As used herein, “culturing the host cell under conditions suitable for the propagation of the vector” means using any cell culture conditions and techniques known in the art which are suitable for the chosen host cell, and which enable the vector to be produced in the host cell. As used herein, “obtaining the vector”, means using any technique known in the art that is suitable for separating the vector from the host cell. Thus, the host cells may be lysed to release the vector. The vector may subsequently be isolated and purified using any suitable method or methods known in the art.

The invention also provides a host cell comprising a nucleic acid molecule, vector or plasmid of the invention.

Preferably, the host cell is a eukaryotic host cell. Examples of eukaryotic host cells include yeast and mammalian cells.

The host cell is preferably a cell in which a vector (e.g. a non-replicating poxvirus vector or an adenovirus vector, as described above) may be grown or propagated. The host cell may be selected from a 293 cell (also known as a HEK, or human embryonic kidney, cell), a CHO cell (Chinese Hamster Ovary), a CCL81.1 cell, a Vero cell, a HELA cell, a Per.C6 cell, a BHK cell (Baby Hamster Kidney), a primary CEF cell (Chicken Embryo Fibroblast), a duck embryo fibroblast cell, or a DF-1 cell.

In other embodiments, the host cell is a human cell (e.g. an isolated human cell).

In a further embodiment, there is provided a virus-like particle (VLP) comprising one or more fusion polypeptides of the invention. The particle is preferably immunogenic.

Virus-like particles resemble viruses, but are non-infectious because they do not contain any viral genetic material. The particles may also be described as multimeric lipoprotein particles.

Once expressed in an appropriate system, these VLPs are able to assemble spontaneously into lipoprotein structures/particles composed of one or more monomers of said fusion polypeptides.

The invention also provides a VLP wherein one or more fusion polypeptides of the invention are covalently attached to the VLP. For example, the fusion polypeptides of the invention may be covalently attached to the VLP by using chemical cross-linkers, reactive unnatural amino acids or SpyTag/SpyCatcher reactions.

The invention also provides a composition comprising a fusion polypeptide of the invention, a nucleic acid molecule of the invention, a vector of the invention or a VLP of the invention, optionally together with one or more pharmaceutically-acceptable carriers, excipients or diluents.

Preferably, the composition is an immunogenic composition.

Substances suitable for use as pharmaceutically-acceptable carriers are known in the art. Non-limiting examples of pharmaceutically-acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long term storage. Examples of buffering agents include, but are not limited to, sodium succinate (pH 6.5), and phosphate buffered saline (PBS; pH 7.4).

In addition to a pharmaceutically-acceptable carrier, the composition of the invention can be further combined with one or more of a salt, excipient, diluent, adjuvant, immunoregulatory agent and/or antimicrobial compound.

In one embodiment, the products of the invention may contain 5% to 95% of active ingredient, such as at least 10% or 25% of active ingredient, or at least 40% of active ingredient or at least 50, 55, 60, 70 or 75% active ingredient.

The products of the invention may be administered in a manner compatible with the dosage formulation, and in such amount as will be prophylactically and/or therapeutically effective.

Administration of the products of the invention is generally by conventional routes, e.g. intravenous, subcutaneous, intraperitoneal, or mucosal routes. The administration may be by parenteral administration; for example, a subcutaneous or intramuscular injection.

Accordingly, the products of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules. The active ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the products of the invention may also contain minor amounts of auxiliary substances such as wetting or emulsifying agents, and/or pH buffering agents.

Additional formulations which are suitable for other modes of administration include oral formulations or formulations suitable for distribution as aerosols. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It may be desired to direct the products of the present invention (as described above) to the respiratory system of a subject. Efficient transmission of a therapeutic/prophylactic composition or medicament to the site of infection in the lungs may be achieved by oral or intra-nasal administration.

Formulations for intranasal administration may be in the form of nasal droplets or a nasal spray. An intranasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

In some preferred embodiments, there is provided a composition comprising a viral vector of the invention, preferably an adenoviral vector of the invention, for intranasal administration.

Alternatively, the therapeutic/prophylactic formulation or medicament may be an aerosol formulation. The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

Preferably, the composition is a vaccine composition, e.g. suitable for parenteral administration, optionally together with one or more adjuvants.

As used herein, a vaccine is a formulation that, when administered to an animal subject such as a mammal (e.g. a human, bovine, porcine, ovine, caprine, equine, cervine, canine or feline subject; in particular a human subject), stimulates a protective immune response against an infectious disease. The immune response may be a humoral and/or a cell-mediated immune response. Thus, the vaccine may stimulate B cells and/or T cells.

Examples of suitable adjuvants include those which are selected from the group consisting of:
metal salts such as aluminium hydroxide or aluminium phosphate,
oil in water emulsions,
toll like receptors agonist, (such as toll like receptor 2 agonist, toll like receptor 3 agonist, toll like receptor 4 agonist, toll like receptor 7 agonist, toll like receptor 8 agonist and toll like receptor 9 agonist),
saponins, for example Quil A and its derivatives such as QS7 and/or QS21,
CpG containing oligonucleotides,
3D-MPL,
(2-deoxy-6-o-[2-deoxy-2-[(R)-3-dodecanoyloxytetra-decanoylamino]-4-o-phosphono-β-D-glucopyranosy]]-2-[(R)-3-hydroxytetradecanoylamino]-α-D-glucopyranosyldihydrogenphosphate),
DP (3S, 9R)-3-[(R)-dodecanoyloxytetradecanoylamino]-4-oxo-5-aza-9(R)-[(R)-3-hydroxytetradecanoylamino] decan-1, 10-diol, 1,10-bis(dihydrogenophosphate), and
MP-Ac DP (3S-, 9R)-3-[(R)-dodecanoyloxytetradecanoylamino]-4-oxo-5-aza-9-[(R)-3-hydroxytetradecanoylamino]decan-I,10-diol, 1-dihydrogenophosphate 10-(6-aminohexanoate), or combinations thereof.

Preferably, the adjuvant is selected from the group comprising:
a saponin associated with a metallic salt, such as aluminium hydroxide or aluminium phosphate
3D-MPL, QS21 and a CpG oligonucleotide, for example as an oil in water formulation,
saponin in the form of a liposome, for example further comprise a sterol such as QS21 and sterol, and
ISCOM.

In some particularly preferred embodiments, the adjuvant comprises a saponin.

Saponins are steroid or triterpenoid glycosides, which occur in many plant species.

Saponin-based adjuvants act in part by stimulating the entry of antigen-presenting cells into the injection site and enhancing antigen presentation in the local lymph nodes.

Preferably, the adjuvant comprises saponin, cholesterol and a phospholipid, e.g. ISCOM Matrix-M™ (Isconova, Novavax).

In Matrix-M, purified saponin fractions are mixed with synthetic cholesterol and a phospholipid to form stable particles than can be readily formulated with a variety of vaccine antigens. Matrix-M™ induces both a cell-mediated and an antibody mediated immune response.

In some other preferred embodiments, the adjuvant comprises a squalene-oil-in-water nano-emulsion emulsion, e.g. AddaVax™ (InvivoGen).

Squalene is an oil which is more readily metabolized than the paraffin oil used in Freund's adjuvants. Squalene oil-in-water emulsions are known to elicit both cellular (Th1) and humoral (Th2) immune responses. This class of adjuvants is believed to act through recruitment and activation of APC and stimulation of cytokines and chemokines production by macrophages and granulocytes.

The composition may further comprise a surfactant. Examples of suitable surfactants include Tween (such as Tween 20), briji and polyethylene glycol.

Vaccine preparation is generally described in New Trends and Developments in Vaccines, edited by Voller et al., University Park Press, Baltimore, Md., U.S.A., 1978. Encapsulation within liposomes is described, for example, by Fullerton, U.S. Pat. No. 4,235,877.

The amount of the fusion polypeptide or particle or nucleic acid molecule of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each dose will comprise 1-1000 μg of protein, for example 1-200 μg, such as 10-100 μg, and more particularly 10-40 μg. An optimal amount for a particular vaccine can be ascertained by standard studies involving observation of antibody titres and other responses in subjects. Following an initial vaccination, subjects will preferably receive a boost in about 4 weeks, followed by repeated boosts every six months for as long as a risk of infection exists. The immune response to the fusion polypeptides of this invention is enhanced by the use of adjuvant and or an immunostimulant.

The amount of saponin for use in the adjuvants of the present invention may be in the region of 1-1000 μg per dose, generally 1-500 μg per dose, more such as 1-250 μg per dose, and more specifically between 1 to 100 μg per dose (e.g. 10, 20, 30, 40, 50, 60, 70, 80 or 90 μg per dose).

The invention also provides a combined preparation comprising two or more components selected the group consisting of fusion polypeptides of the invention, particles of the invention, nucleic acids of the invention, vectors of the invention and compositions of the invention, as a combined preparation in a form suitable for simultaneous, separate or sequential use, preferably for treating or preventing Staphylococcus aureus infection.

As used herein, the term “product of the invention” refers to the fusion polypeptides of the invention, particles of the invention, nucleic acids of the invention, vectors of the invention, antibodies of the invention and compositions of the invention.

In yet another aspect, the invention provides an antibody against a fusion polypeptide of the invention, wherein the antibody:
(a) disrupts or prevents binding of EapH1 to EapH2 or to Eap; or
(b) disrupts or prevents binding of EapH2 to EapH1 or to Eap.

In some embodiments, the antibody does not bind to an EapH1 polypeptide of SEQ ID NO: 2 or 4. In some embodiments, the antibody does not bind to an EapH2 polypeptide of SEQ ID NO: 6 or 8. In some embodiments, the antibody does not bind to an Eap polypeptide of SEQ ID NO: 9.

The antibody is preferably a monoclonal antibody.

In yet further embodiments, the invention provides a fusion polypeptide of the invention, a particle of the invention, an antibody of the invention, a nucleic acid of the invention, a vector of the invention or a composition of the invention, for use in therapy or for use as a medicament.

In a further aspect, the invention provides a fusion polypeptide of the invention, a particle of the invention, an antibody of the invention, a nucleic acid of the invention, a vector of the invention or a composition of the invention for use in a method of preventing or treating a Staphylococcus aureus infection in a subject.

In particular, the invention provides a fusion polypeptide of the invention, a particle of the invention, an antibody of the invention, a nucleic acid of the invention, a vector of the invention or a composition of the invention for use in a method of preventing kidney abscesses due to a Staphylococcus aureus infection in a subject.

In particular, the invention provides a fusion polypeptide of the invention, a particle of the invention, an antibody of the invention, a nucleic acid of the invention, a vector of the invention or a composition of the invention for use in a method of reducing carriage of Staphylococcus aureus bacteria in a subject.

In a further aspect, the invention provides a fusion polypeptide of the invention, a particle of the invention, an antibody of the invention, a nucleic acid of the invention, a vector of the invention or a composition of the invention for use in a method of inducing a T-cell response or a B-cell response to a Staphylococcus aureus antigen in a subject.

In particular, a non-replicating poxvirus vector of the invention can be used to stimulate a protective immune response via the cell-mediated immune system. In one embodiment, the T-cell is a T-helper cell (Tₕ-cell). In one embodiment, the T-cell is a Tₕ17-cell.

In further embodiments, the invention provides the use of a fusion polypeptide of the invention, a particle of the invention, an antibody of the invention, a nucleic acid of the invention, a vector of the invention or a composition of the invention in the manufacture of a medicament for use in a method of preventing or treating a Staphylococcus aureus infection in a subject.

In further embodiments, the invention provides the use of a fusion polypeptide of the invention, a particle of the invention, an antibody of the invention, a nucleic acid of the invention, a vector of the invention or a composition of the invention in the manufacture of a medicament for use in a method of preventing kidney abscesses due to a Staphylococcus aureus infection in a subject.

In further embodiments, the invention provides the use of a fusion polypeptide of the invention, a particle of the invention, an antibody of the invention, a nucleic acid of the invention, a vector of the invention or a composition of the invention in the manufacture of a medicament for use in a method of reducing carriage of Staphylococcus aureus bacteria in a subject.

In further embodiments, the invention provides the use of a fusion polypeptide of the invention, a particle of the invention, an antibody of the invention, a nucleic acid of the invention, a vector of the invention or a composition of the invention in the manufacture of a medicament for use in a method of inducing a T cell response or a B-cell response to a Staphylococcus aureus antigen in a subject.

The invention also provides a method of treating a subject susceptible to Staphylococcus aureus infection comprising administering an effective amount of a fusion polypeptide of the invention, a particle of the invention, an antibody of the invention, a nucleic acid of the invention, a vector of the invention or a composition of the invention, to the subject.

The invention also provides a method of preventing kidney abscesses due to a Staphylococcus aureus infection in a subject comprising administering an effective amount of a fusion polypeptide of the invention, a particle of the invention, an antibody of the invention, a nucleic acid of the invention, a vector of the invention or a composition of the invention, to the subject.

The invention also provides a method of reducing carriage of Staphylococcus aureus bacteria in a subject comprising administering an effective amount of a fusion polypeptide of the invention, a particle of the invention, an antibody of the invention, a nucleic acid of the invention, a vector of the invention or a composition of the invention, to the subject.

The invention also provides a method of inducing a T-cell response or a B-cell response to a Staphylococcus aureus antigen in a subject comprising administering an effective amount of a fusion polypeptide of the invention, a particle of the invention, an antibody of the invention, a nucleic acid of the invention, a vector of the invention or a composition of the invention to the subject.

A fusion polypeptide of the invention, a particle of the invention, an antibody of the invention, a nucleic acid of the invention, a vector of the invention or a composition of the invention may also be used in similar uses and methods to produce neutralising antibodies in vivo against Staphylococcus aureus antigens.

The efficacy of the uses and methods to treat/prevent Staphylococcus aureus infection may be tested (e.g. by ELISA) by establishing the presence or absence of neutralising antibodies against Staphylococcus aureus bacteria in the subject's blood.

The subject is preferably a mammal, e.g. a human, pig, cow or horse, more preferably a human.

S. aureus carriage is a highly important factor in S. aureus transmission and increases the risk of development of invasive disease. The fusion polypeptides, particles, nucleic acid molecules, vectors and compositions of the invention can be used to treat individuals carrying S. aureus, such that the number of S. aureus bacteria present on or in the individual is reduced (for example, by 50, 60, 70, 80 or 90%, as compared to prior to treatment) or effectively eliminated (for example, by reducing the number of S. aureus bacteria present on or in the individual by greater than 99%, such as 99.5 or 99.9 or 99.99%), as compared to prior to treatment).

As used herein, the term “preventing” includes preventing the initiation of Staphylococcus aureus infection and/or reducing the severity of intensity of a Staphylococcus aureus infection. Thus, “preventing” encompasses vaccination.

As used herein, the term “treating” embraces therapeutic and preventative/prophylactic measures (including post-exposure prophylaxis) and includes post-infection therapy and amelioration of a Staphylococcus aureus infection. Each of the above-described methods and uses can comprise the step of administering to a subject an effective amount, such as a therapeutically effective amount, of a fusion polypeptide of the invention, a particle of the invention, a nucleic acid of the invention, a vector of the invention or a composition of the invention.

As used herein, an effective amount is a dosage or amount that is sufficient to achieve a desired biological outcome. As used herein, a therapeutically effective amount is an amount which is effective, upon single or multiple dose administration to a subject (such as a mammalian subject, in particular a human subject) for treating, preventing, curing, delaying, reducing the severity of, ameliorating at least one symptom of a disorder or recurring disorder, or prolonging the survival of the subject beyond that expected in the absence of such treatment.

Accordingly, the quantity of active ingredient to be administered depends on the subject to be treated, capacity of the subject's immune system to generate a protective immune response, and the degree of protection required. Precise amounts of active ingredient required to be administered may depend on the judgement of the practitioner and may be particular to each subject. Administration to the subject can comprise administering to the subject a fusion polypeptide of the invention, a particle of the invention, a nucleic acid of the invention, a vector of the invention or a composition of the invention (i.e. a product of the invention) wherein the product of the invention is sequentially administered multiple times (for example, wherein the composition is administered two, three or four times). Thus, in one embodiment, the subject is administered a fusion polypeptide of the invention, a particle of the invention, a nucleic acid of the invention, a vector of the invention or a composition of the invention and is then administered the same product of the invention (or a substantially similar product) again at a different time.

In one embodiment, administration to a subject comprises administering a fusion polypeptide of the invention, a particle of the invention, a nucleic acid of the invention, a vector of the invention or a composition of the invention to a subject, wherein said said product of the invention is administered substantially prior to, simultaneously with, or subsequent to, another immunogenic composition.

The invention also extends to prime-boost regimes. For example, priming and/or boosting may be effected using one or more products of the invention. The products may be administered to a subject sequentially, simultaneously or separately.

In one embodiment, the first and second products are administered as part of a prime-boost administration protocol. Thus, the first product may be administered to a subject as the “prime” and the second product subsequently administered to the same subject as the “boost”.

In one embodiment, the first product is an adenovirus vector of the invention prime, and the second product is a non-replicating poxvirus vector of the invention boost.

In one embodiment, each of the above-described methods further comprises the step of administration to the subject of a product of the invention.

In one embodiment, the fusion polypeptide of the invention is administered separately from the administration of a viral vector of the invention. Preferably the fusion polypeptide and the viral vector are administered sequentially, in any order. Thus, in one embodiment, the viral vector (“V”) and the fusion polypeptide (“P”) may be administered in the order V-P, or in the order P-V.

In other embodiments, the fusion polypeptide and viral vector are admixed, and are administered together to the subject.

In certain embodiments, the above-described methods further comprise the administration to the subject of an adjuvant. Adjuvant may be administered with any of the products of the invention.

The products of the invention may be given in a single dose schedule (i.e. the full dose is given at substantially one time). Alternatively, the products of the invention may be given in a multiple dose schedule.

A multiple dose schedule is one in which a primary course of treatment (e.g. vaccination) may be with 1-6 separate doses, followed by other doses given at subsequent time intervals required to maintain and or reinforce the immune response, for example (for human subjects), at 1-4 months for a second dose, and if needed, a subsequent dose(s) after a further 1-4 months.

The dosage regimen will be determined, at least in part, by the need of the individual and be dependent upon the judgment of the practitioner (e.g. doctor or veterinarian).

Simultaneous administration means administration at (substantially) the same time.

Sequential administration of two or more products of the invention means that the products are administered at (substantially) different times, one after the other.

For example, sequential administration may encompass administration of two or more products of the invention at different times, wherein the different times are separated by a number of days (for example, 1, 2, 5, 10, 15, 20, 30, 60, 90, 100, 150 or 200 days).

For example, in one embodiment, the vaccine of the present invention may be administered as part of a ‘prime-boost’ vaccination regime.

In one embodiment, the products of the invention can be administered to a subject such as a mammal (e.g. a human, bovine, porcine, ovine, caprine, equine, cervine, canine or feline subject) in conjunction with (simultaneously or sequentially) one or more immunoregulatory agents selected from, for example, immunoglobulins, antibiotics, interleukins (e.g. IL-2, IL-12), and/or cytokines (e.g. IFN-γ).

In yet further embodiments, the invention provides a process for the production of a particle or fusion polypeptide of the invention, which process comprises expressing a nucleic acid molecule coding for said particle or polypeptide in a suitable host and recovering the product. Preferably, the host is a human cell.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997), supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

In some embodiments, tblastn (NCBI Blast suite v. 2.4.0) may be used using default parameters. Ungapped matches to more than 80% of the query with e<10⁻¹⁰ are considered significant.

In some embodiments, the NCBI RefSeq database may be queried using BLASTp and delta-BLAST using default parameters. Alignments may be prepared using the NCBI Cobalt multiple alignment engine with default parameters.

Sequences are most preferably aligned pairwise using the Needleman-Wunsch global sequence alignment algorithm (Needleman, Saul B. & Wunsch, Christian D. (1970). “A general method applicable to the search for similarities in the amino acid sequence of two proteins”. Journal of Molecular Biology. 48 (3): 443-53. doi:10.1016/0022-2836(70)90057-4. PMID 5420325.) as implemented in the nwalign 0.3.1 python package https://pypi.python.org/pypi/nwalign. Alignments are made with a gap opening penalty of 10 and an extension penalty of 4 using the BLOSUM62 matrix (Henikoff, J. G. Amino acid substitution matrices from protein blocks. Proc. Natl. Acad. Sci. USA 89, 10915-10919 (1992)) obtained from the National Centres for Biotechnology Information. Preferably, for sequence “identity”, at each position in the alignment, a score of 1 is given if the amino acids are identical; zero is assigned in all other positions, including those with gaps.

The disclosure of each reference set forth herein is incorporated herein by reference in its entirety.

### BRIEF DESCRIPTION OF THE FIGURES

FIG. 1: Sequence diversity of EapH proteins in 104 S. aureus isolates

The presence and conservation of EapH1, EapH2, and comparator proteins in a set of 104 sequenced clinical S. aureus isolates representative of major S. aureus clones was determined by tblastn against de novo assemblies of Illumina sequencing of the clones. N=x/104 indicates that a homologue matching the query over 95% of the protein length was found in x sequences.

FIG. 2: Interaction between EapH proteins and Eap

Recombinant EapH and Eap proteins were produced from E. coli, and a C-terminal 6-HIS tag was either cleaved off or retained. Cleaved 6-HIS tags were removed by dialysis (A). Untagged proteins were immobilised on ELISA plates and His-Tagged partners applied at a range of concentrations (B-D). Following washing, detection of binding of a His-tagged ligand was detected by anti-His alkaline phosphatase polyclonal antibody (B). The extracellular domains of SAUSA300_2132 and SAUSA300_1795, two cell surface S. aureus proteins, were immobilised on the plate as negative controls. A model of the complex predicted is shown in E.

FIG. 3: Production of recombinant EapH proteins from viral vectors

Domains from the physiological Eap, EapH1, and EapH2 proteins (A) were expressed from adenovirus and modified Vaccinia Ankara (MVA) vectors behind a mammalian signal sequence, and fused to V5 epitope tag and IMX313 multimerising domain (B). V5 and IMX313 tagged proteins were detected from both supernatant (C) and cell lysates (D) following infection of HeLa cells with adenoviruses expressing these constructs.

FIG. 4: Immunogenicity of Adenovirus-MVA regimes expressing EapH proteins

Balb/c mice were vaccinated with adenovirus Hu5 expressing no antigen (‘control’) or expressing EapH1_2, Eap domain 5, or EapH1_2_Eap constructs. Specific antibody against (B) EapH1 and (C) EapH2 were determined by LIPS on day 70. Anti-Eap end-point titres were determined on a random subset of animals from each group by ELISA (D). 3 days following i.v. challenge, the number of abscesses in the right kidney was determined by post-mortem MRI (E), and bacterial recovery from the left kidney determined (F).

FIG. 5: Protection following Adenovirus-MVA regimes expressing EapH1 and H2

Balb/c mice were vaccinated with adenovirus Hu5 expressing no antigen (control) or EapH1_2 antigen. The regime is identical to that shown in FIG. 4, and the experiment shown in FIG. 4 is summarised as experiment 1 here, along with two further larger experiments. Bacterial recovery from left kidney (A). Associations between renal bacterial load and peripheral blood anti-EapH1/H2 γ-IFN ELISPOT (B), anti-EapH1 antibody (C) and anti-EapH2 antibody (D) on day 70.

FIG. 6: Intranasal vaccination with vectors expressing EapH1 and H2

CD1 mice were vaccinated intranasally with a single dose of a viral vector (AdHu5 or MVA) vector expressing EapH1 and EapH2, or a control. Serological responses were monitored in tail bleeds. (A). Antibody responses against EapH1 and EapH2 were measured (B, C). 26 days after vaccination, mice were exposed to S. aureus by environmental contamination, and carriage of S. aureus monitored. Carriage levels at 1 day (D) and 28 days (E) after S. aureus exposure.

FIG. 7: Human serological responses to EapH1 and EapH2

IgG recognising EapH1, EapH2 and four control antigens (the cell surface proteins IsdA, IsdB, ClfB and the nuclease Nuc1) was quantified in a cohort of 42 humans, randomly selected from cohort studies, including 19 carriers (identified by having two S. aureus nasal swabs positive) and 23 non-carriers (A). Antibodies against EapH1 and EapH2 were also quantified (B). Correlations between antibody titres from the 4 control antigens, EapH1 and EapH2 with significant correlations (Spearman's rho differs from 0, p<0.01) indicated by plotting of a regression line (C). Carrier and non-carrier human EapH1 (D) and EapH2 (E) antibody titres relative to responses to control antigens (IsdA, IsdB, ClfB, Nuc1) were measured.

FIG. 8: Interaction between EapH proteins and Eap

Recombinant EapH and Eap proteins were produced from HEK293 cells, fused to either a C-terminal V5 tag or to Renilla luciferase. V5 tagged proteins were captured onto anti-V5 coated plate, and Renilla luciferase-ligands incubated at a range of concentrations.

FIG. 9: Stool carriage following intramuscular viral vector vaccination

Balb/c mice were vaccinated with adenovirus H5 expressing no antigen (control) or EapH1_2 antigen. Data on immunogenicity and impact on bacterial load is shown in FIG. 5. The regime is identical to that shown in FIG. 4, and the experiment shown in FIG. 4 is summarised as experiment 1 here, along with two further larger experiments. Effect of vaccination on proportion of mice with positive S. aureus bacterial recovery from stool is shown for the three experiments.

FIG. 10: Stool carriage following intranasal viral vector vaccination

CD1 mice were vaccinated intranasally with a single dose of a viral vector (AdHu5 or MVA) vector expressing EapH1 and EapH2, or a control. The experiment consisted of eight cages of mice; one mouse per cage received a different treatment. Stool counts in mice receiving adenovirus expressing either Control or EapH1_2 are shown, following exposure to S. aureus by environmental contamination.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Materials and Methods

### Bioinformatic Analyses

Velvet-assembled contigs from Illumina next-generation sequencing of a collection of S. aureus strains [26] were interrogated by tblastn (NCBI Blast suite v. 2.4.0) using default parameters. Ungapped matches to more than 80% of the query with e<10⁻¹⁰ were considered significant.

### Bacterial Strains and Growth

Staphylococcus aureus strain Newman was kindly provided by Prof. T. Foster, Trinity College, Dublin (Ireland). For infection of mice, S. aureus Newman were grown on Horse Blood Agar (HBA, Oxoid, UK), three colonies were inoculated into 10 mL tryptic soy broth (TSB, Oxoid, UK) and grown overnight at 37° C., 130 rpm. The resulting culture was subcultured 1:100 into 10 mL of fresh TSB and incubated statically at 37° C. for 2.5 h. Bacteria were harvested by centrifugation, washed once and then resuspended in 10 mL Phosphate Buffered Saline (PBS, Sigma Aldrich, UK) at approximately 1×10⁸ cells/mL. The actual bacterial concentrations were determined by dilution plating.

### Antigens and Viral Vectors

Vaccine ‘Eap’ contained C-terminal MAP domain of S. aureus Eap protein with an adjacent C-terminal basic region (a. a. 481-584 of WP_001549158.1); vaccine ‘EapH_1_2’ contained MAP domains of EapH1 (a. a. 24-141 of WP_001549607.1) and EapH2 (a. a. 24-144 of WP_000769689.1); vaccine ‘Eap_EapH1_EapH2’ was composed of all three antigens (Table 1). DNA sequences encoding vaccine constructs were human codon optimized and combined with Human Tissue Plasminogen Activator leader signal sequence (TPA), V5 epitope tag and IMX313 oligomerization domain (Spencer et al, 2012). DNA strings were synthesised by Life Technologies Ltd. using GeneArt Gene Synthesis and subcloned by restriction digest into a pMono2 mammalian expression vector (WO2014/053861A2). For Adenovirus vaccines antigen constructs or pMono2 backbone (for control vaccine) were subcloned from pMono2 into pAdH5-PL-pDEST shuttle vector using Gateway technology (Life Technologies), linearized by PacI and transfected into replication-deficient adenovirus human serotype 5 (AdHu5) as described elsewhere (Draper et al., 2008; Gilbert et al., 2002). For Modified Vaccinia Ankara (MVA) vaccines, antigens or pMono2 backbone were transferred into an MVA shuttle vector by restriction digestion, linearized with AatII and transfected into MVA (Draper et al., 2008; Gilbert et al., 2002). All inserts were confirmed by sequencing.

TABLE 1

| | | | | log2 fold change |
|---|---|---|---|---|
| | | | | in protein |
| | | | | concentration in |
| | | | Amino acids | supernatant in |
| | | USA300 | used in | rsp mutants of |
| Protein | NCBI | Locus id | vaccine | USA300 |
| Eap | WP_001549158.1 | Map | 481 . . . 584 | ND |
| | | (pseudogene) | | |
| EapH1 | WP_001549607.1 | SAUSA300_2614 | 24 . . . 141 | 3.53 |
| EapH2 | WP_000769689.1 | SAUSA300_0883 | 24 . . . 144 | 3.83 |

### Testing Antigen Production by Viral Vectors

HeLa cells we seeded at 10⁵ cells per well on the 24 well plate and allowed to grow overnight. The media in the wells was replaced by 2×10⁸, 2×10⁷ or 2×10⁶ I.U. of adenovirus preparation (in 250 μl) and incubated for 2 h. Further 250 μl of media was added to the wells and the plates were incubated overnight. All incubation steps were done at 37° C., 5% COO₂. 96-well NUNC plate was coated with 1:500 dilution of mouse anti-V5 antibody (Abcam) in PBS, blocked with 1% BSA/PBS and incubated with 50 μl of supernatant from adenovirus-infected HeLa cells. They were further incubated with 1:1000 dilution of serum from rabbits immunized with IMX313 protein (kindly provided by IMAXIO) and 1:500 dilution of the goat anti-rabbit IgG HRP conjugated antibody (Jackson ImmnoResearch). Plates were washed with PBS/0.05% Tween after each incubation, developed with TMB Substrate Solution (ThermoFisher), stopped with 2M H₂SO₄ and read at 450 nm.

### Recombinant Protein Production

DNA strings encoding C-terminal MAP domain and adjacent basic region of Eap and MAP domains of EapH1 and EapH2 with N-terminal His-tag and 3C protease site were ordered from GeneArt Gene Synthesis (ThermoFisher) (Table 2). DNA strings were inserted into the pOPIN-F vector (kindly provided by the Dr Ray Owens, Oxford University, Oxford, UK) via their complimentary sites using In-Fusion reaction (Clontech). The resulting plasmids were transformed into DH5a Competent cells (Thermofisher). Plasmids were purified from transformed cells using QIAprep Spin Miniprep Kit (QIAGEN) and insert sequence was validated by Sanger sequencing using T7 forward and TriEx reverse primers. Purified plasmids were transformed into One Shot BL21 Star (DE3) Chemically Competent E. coli cells according to the manufacturer's protocol. Protein expression was done using Overnight Express Autoinduction System 1 (Novagen). Proteins were purified using BugBuster Ni-NTA His*Bind Purification Kit (Merck Millipore) and dialysed against PBS using Slide-A-Lyzer G2 Dialysis Cassettes, 7K MWCO (Thermofisher). On average 2 to 10 mg of protein was obtained from 50 ml culture.

TABLE 2
Sequences synthesised

| Gene | Sequence used in viral vector |
|---|---|
| Eap | aagttctgtttcagggcccgGTTCCGTACACCATCGCGGTTAACGGTACCTCTACCCCGA |
| SEQ ID | TCCTGTCTAAACTGAAAATCTCTAACAAACAGCTGATCTCTTACAAATACCTGAACGACA |
| NO: 15 | AAGTTAAATCTGTTCTGAAATCTGAACGTGGTATCTCTGACCTGGACCTGAAATTCGCGA |
| | AACAGGCGAAATACACCGTTTACTTCAAAAACGGTAAAAAACAGGTTGTTAACCTGAAAT |
| | CTGACATCTTCACCCCGAACCTGTTCTCTGCGAAAGACATCAAAAAAATCGACATCGACG |
| | TTAAACAGTACACCAAATCTAAAAAAAACAAAtaaagctttctagaccat |
| EapH1 | aagttctgtttcagggcccgCCGTCTCACGAAGCGTCTGCGGACTCTAACAACGGTTACA |
| SEQ ID | AAGAAATGACCGTTGACGGTTACCACACCGTTCCGTACACCATCTCTGTTGACGGTATCA |
| NO: 16 | CCGCGCTGCACCGTACCTACTTCATCTTCCCGGAAAACAAAAACGTTCTGTACCAGGAAA |
| | TCGACTCTAAAGTTAAAAACGAACTGGCGTCTCAGCGTGGTGTTACCACCGAAAAAATCA |
| | ACAACGCGCAGACCGCGACCTACACCCTGACCCTGAACGACGGTAACAAAAAAGTTGTTA |
| | ACCTGAAAAAAAACGACGACGCGAAAAACTCTATCGACCCGTCTACCATCAAACAGATCC |
| | AGATCGTTGTTAAAtaaagctttctagaccat |
| EapH2 | aagttctgtttcagggcccgGCGGGTAACGAAGTTTCTGCGGCGGAAAAAGACAAACTGC |
| SEQ ID | CGGCGACCCAGAAAGCGAAAGAAATGCAGAACGTTCCGTACACCATCGCGGTTGACGGTA |
| NO: 17 | TCATGGCGTTCAACCAGTCTTACCTGAACCTGCCGAAAGACTCTCAGCTGTCTTACCTGG |
| | ACCTGGGTAACAAAGTTAAAGCGCTGCTGTACGACGAACGTGGTGTTACCCCGGAAAAAA |
| | TCCGTAACGCGAAATCTGCGGTTTACACCATCACCTGGAAAGACGGTTCTAAAAAAGAAG |
| | TTGACCTGAAAAAAGACTCTTACACCGCGAACCTGTTCGACTCTAACTCTATCAAACAGA |
| | TCGACATCAACGTTAAAACCAAAtaaagctttctagaccat |

### Murine Renal Abscess Model

For the experiments in this study, a total of 127 weight-matched female BALB/C mice, aged 6 weeks were obtained from Harlan Laboratories (Bicester, UK). Mice were housed randomly in cages of 3, 4 or 6. To determine the protective effect of the vaccines we used a murine intravenous challenge model. Treatment groups were allocated such that each cage contained at least one animal receiving each treatment. Mice received an intramuscular injection with 10⁹ IU AdHu5 expressing vaccine antigen or no antigen, followed 10 weeks later with a boost vaccination comprising 10⁷ PFU MVA expressing either the vaccine antigen or GFP. Two weeks after the boost vaccination mice were challenged with ˜10⁷ CFU S. aureus Newman bacterial suspension in 0.1 ml PBS injected into the lateral tail vein. Mice were weighed and monitored daily for signs of illness. Three days post infection mice were sacrificed and kidneys and spleens were harvested. The left kidney was homogenised in PBS, plated and viable bacteria per gram tissue were counted. Viable S. aureus per gram of tissue/ml of blood were enumerated by spreading serial diluted aliquots of homogenized tissue (GentleMACS, M-tubes, Miltenyi Biotec, Bisley, UK) for colony formation using an Autoplate machine (Quadrachem, UK) on horse blood agar (HBA) (Oxoid). Plates were incubated for 24 hours at 37° C. in air, and colonies were counted using an automated counter (QCount, Quadrachem, UK).

### Abscess Formation

In addition to enumerating bacterial recovered from kidneys, abscess formation was examined using Magnetic Resonance Imaging (MRI) of post-mortem material. Briefly, after mice were sacrificed the right kidney was fixed and stored in 4% paraformaldehyde (Alfa Aesar, UK). MRI was performed as described [27]. Image analysis was performed using Amira software version 5.6 (FEI Visualisation Sciences Group). Kidneys were analysed for total abscess volume, individual abscess volumes and the number of abscesses detected in each kidney, and compared across treatment groups.

### Intranasal Vaccination and Experimental Colonisation of Mice

40 female CD1 mice, aged 6 weeks were obtained from Envigo (Netherlands). Mice were housed randomly in cages of 5. Treatment groups were allocated such that each cage contained at one animal receiving each treatment. Mice received 100 μl intranasal dose of PBS, 10⁹ IU AdHu5 expressing vaccine antigen or no antigen, or 10⁷ PFU MVA expressing either the vaccine antigen or GFP. To assess efficacy of vaccines in reducing S aureus carriage, we experimentally colonised mice 26 days after vaccination. S. aureus was prepared by overnight culture in TSB (Oxoid) at 37° C., 130 rpm, washed and resuspended in PBS (Sigma). Contamination of cages was performed by spraying this inoculum onto bedding using a 100 ml plastic spray bottle with a hand-pumped vaporiser (product 215-3092, VWR International). Each cage received 5-10 ml of S. aureus culture at ˜5×10⁹ cfu/ml. Mice were not sprayed directly, and cages were not cleaned for 7 days after spraying.

Evaluation of S. aureus Carriage in Mice

Naturally and experimentally colonised mice were screened for gut carriage of S. aureus during the course of the experiments. Stool samples were taken on arrival, at various time points during the experiment. Stools were weighed and homogenised in sterile PBS (Sigma Aldrich, UK) before plating on Brilliance Staph 24 agar (Oxoid, UK) to determine CFU/g stool. Negative samples were enriched in 5% salt meat broth (Oxoid, UK) at 37° C., 130 rpm, for at least 24 h before plating on Brilliance Staph 24 agar. Samples negative upon enrichment were considered as negative in the analysis.

### Luciferase Immunoprecipitation System (LIPS) Assay

The antibody response to vaccination in mice was assessed by the LIPS assay as previously described (van Diemen et al., 2013; Burbelo et al., 2005). The fusion of the target antigen and Renilla luciferase was expressed in HEK293 cells. Antigen-Renilla luciferase fusion proteins were combined with serially diluted mouse sera obtained one day before challenge. After 1 h incubation at room temperature the mixture was transferred into MultiScreen_{HTS} HV opaque 0.45 μm filter plates (EMD Millipore) containing 3% Protein A/G UltraLink Resin (ThermoFisher). After 1 h incubation and subsequent washings with Buffer A (10 mM Tris, 100 mM NaCl, 5 mM MgCl₂0.6 H₂O, 1% Triton X-100) and PBS (Sigma Aldrich, UK) Renilla luciferase assay reagent (Renilla luciferase assay system, Promega, UK) was added and chemoluminiscence was measured using CLARIOstar microplate reader (BMG LABTECH). Log transformation was applied to luminescence data before subtracting the assay background.

### IFN-γ Enzyme-Linked ImmunoSpot (ELISPOT) Assay

Individual mouse peripheral blood samples were treated with ACK lysis buffer to remove RBCs prior to stimulation with relevant peptides spanning the Eap, EapH1 or EapH2 portions of the immunogen (final concentration of 2 μg/ml) in the presence of homologeous splenocytes (5×10⁶ cells/ml), on High Protein Binding Immobilon-P membrane plates (MAIPS4510, Millipore) coated with 5 mg/ml anti-mouse IFN-γ (AN18, Mabtech). After 18-20 hours, IFN-γ spot forming cells (SFC) were visualised by staining membranes with anti mouse IFN-γ biotin (1 μg/ml, R4-6A2, Mabtech) followed by streptavidin-Alkaline Phosphatase (1 μg/ml, Mabtech) and development with AP conjugate substrate kit (BioRad, UK). The number of SFC were counted with an ELISpot reader (AID, Germany). Log(SFC) was used in statistical analysis because of the approximate log-normal distribution of ELISpot counts in the animals [28].

### End-Point Titre ELISA

96-well NUNC ELISA plate was coated with 0.05 μg/well of recombinant Eap protein diluted in PBS; and blocked with 1% BSA/PBS/0.05% Tween. Further wells were incubated with post-boost sera from 5 mice for each of the vaccination regimens, serially diluted 1:3 in blocking buffer with the lowest dilution of 1:100. Sera from naïve SOPF Balb/C mice were used for background. Goat anti-mouse IgG Alkaline Phosphatase conjugated secondary antibody (Abcam) was used for detection, diluted 1:10,000 in blocking buffer. Plates were washed with PBS/0.05% Tween after each incubation, developed with SIGMAFAST p-Nitrophenyl phosphate tablets (Sigma) and read at 405 nm. After Background subtraction and log-transformation of the data, non-linear regression (dose-response stimulation; log(agonist) vs response-variable slope 4 parameters) was used to fit the curve and interpolate the end-point titre value using GraphPad Prism Software.

### Interaction Assays

For this assay, His tag was excised from the Eap, EapH1 and EapH2 proteins using Pierce HRV 3C Protease Solution Kit (Thermofisher). 96-well NUNC plates were coated with 0.1 mM/well of Eap, EapH1 and EapH2 proteins with His tag excised, diluted in PBS. Plates were blocked with 2% BSA/PBS/0.05% Tween and incubated with PBS (as background) or His-tagged Eap, EapH1, EapH2, SAUSA300_2132 and SAUSA300_1795 at a range from 1,000 to 0.5 mM per well, diluted in PBS. The latter two staphylococcal proteins were used as negative controls. His-tagged proteins alone were coated on the plate as positive controls. Plate was incubated with mouse anti-Histidine tag antibody, Alkaline Phosphatase conjugated (Biorad) diluted 1:1000 in 1% BSA/PBS/0.05% Tween. As a positive control for successful coating some wells were incubated with post-boost sera from EapH1_EapH2 vaccinated mice and goat anti-mouse IgG, Alkaline Phosphatase conjugated (Abcam) secondary antibody. Plates were washed with PBS/0.05% Tween after each incubation, developed with SIGMAFAST p-Nitrophenyl phosphate tablets (Sigma) and read at 405 nm. After background subtraction, non-linear regression with least squares fit was used to create a fitting curve and interpolate K_{d} using GraphPad Prism Software.

IgG Antibody to S. aureus Antigens in Human Cohorts

Forty-two serum samples randomly selected out of two cohorts of healthy adult volunteers (18 to 60 years old) were screened for antibodies against selected S. aureus antigens using LIPS, as described above. Linearity of the response was determined by including a standard curve comprising serial dilutions of human serum from a single, healthy donor. Sera were examined at 1:1000 dilution in PBS. Specific informed consent was obtained from both cohorts, as described by Whitehouse et al. [29]. Briefly, the Oxford Staphylococcus aureus carriage cohort (n=26) comprises healthy adult volunteers from 18 to 60 years old, declaring themselves to be of North European ancestry. Exclusion criteria were pregnancy, intake of immunomodulatory drugs, cancer, connective tissue disease, blood born viruses, or previous organ transplantation. The Submarine cohort (n=49) comprises healthy male submariners who gave serum at the start of a submarine patrol serum were taken, separated within 4 hours of sampling and stored at −80° C. For both cohorts two nasal swabs were taken at least 1 month apart, and cultured both directly on selective agar (BD Brilliance Staph 24) and by enrichment culture in Mannitol salt broth followed by plating on selective agar. Individuals who cultured positive at both time points were considered to be S. aureus carriers, and all others non-carriers. Specific informed consent was obtained from both cohorts, as described [29][30]. From these two studies, serum samples were selected randomly, having stratified for carriage status. Analysis was performed blinded on serum from 19 carriers and 23 non-carriers.

### Statistical Analysis

Data on antibody response and IFN-γ-specific cell numbers were statistically analysed for effect of vaccine by means of t-tests after a log₁₀ transformation and correction for background. Specific antibody levels from the LIPS assay were expressed as fold increase over assay background by subtracting the log transformed assay luminescence background, which was considered to be the luminescence observed in the absence of any sera. The assay limit of detection was considered to be four standard deviations above the background. Post-hoc pairwise comparisons were performed using Dunnett's Multiple Comparison Test. For estimation of the association between S. aureus carriage and antibody titres, a general linear model fitting

yᵢ=Pᵢ+C

where yᵢ=log(fold increase in antibody concentration relative to background)
Pᵢ=the i^{th} protein examined and
C=effect of carriage vs. non-carriage.

Differences were considered significant when p<0.05. The statistical packages used were R 2.15 (http://www.cran.org), and GraphPad Prism version 5.04 (GraphPad Software, Inc.).

### Example 2: Prevalence of EapH1 and EapH2 Across S. aureus Lineages

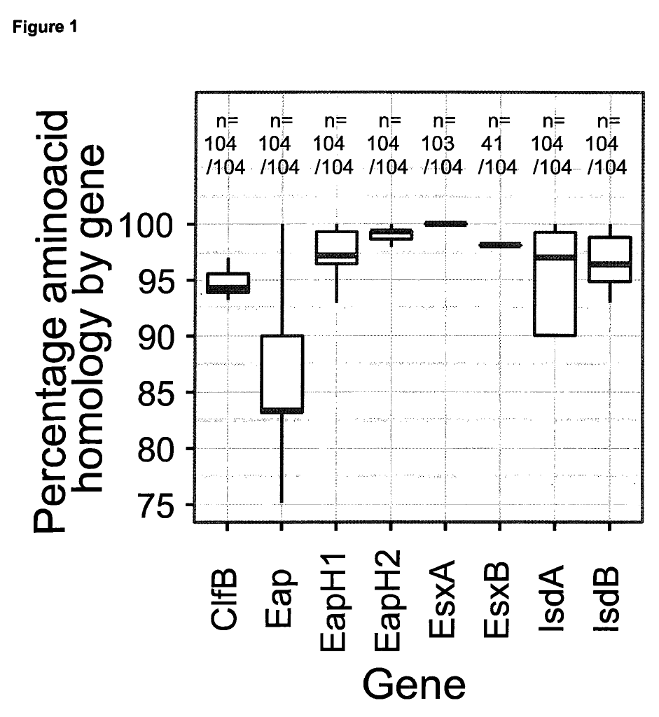

We examined the presence of EapH1 and EapH2 proteins in a set of 104 sequenced clinical S. aureus isolates previously shown to be representative of major S. aureus clones [26]. In common with other recognised core S. aureus genes, such as IsdA and IsdB, we observed EapH1 and EapH2 homologues in all strains examined (FIG. 1). Genetic diversity relative to the query sequences used (WP_001549607.1, WP_000769689.1 respectively) was significantly greater (median 97%) for EapH1 than for EapH2 (median 99%). However, both genes are very highly conserved.

### Example 3: Interactions Between EapH1, EapH2 and C-Terminus of Eap

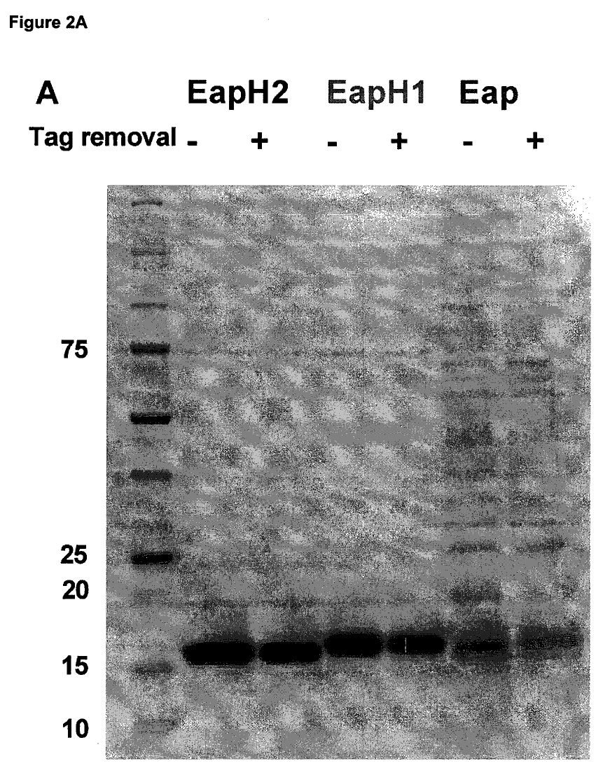

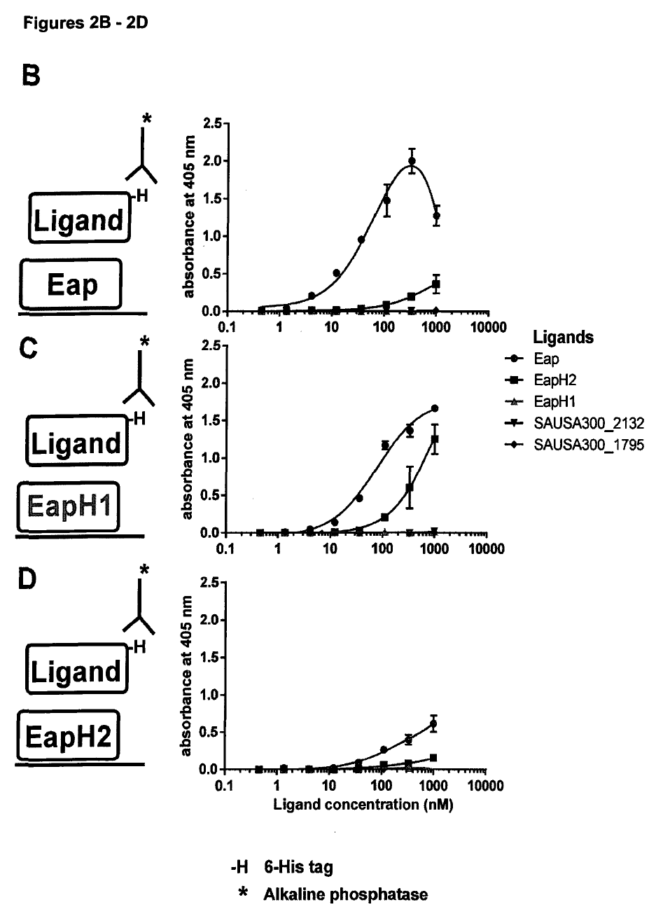

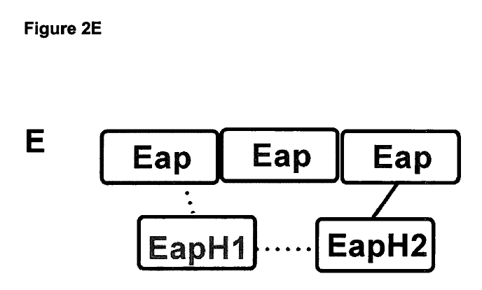

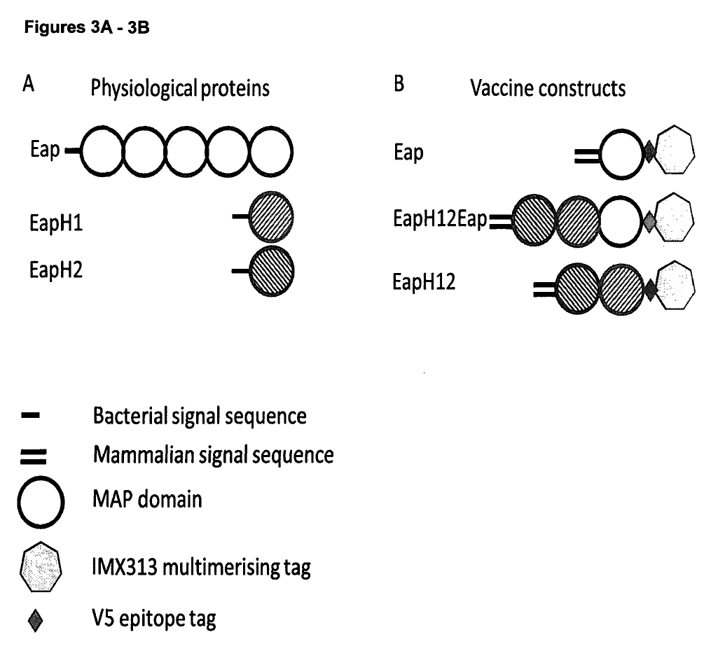

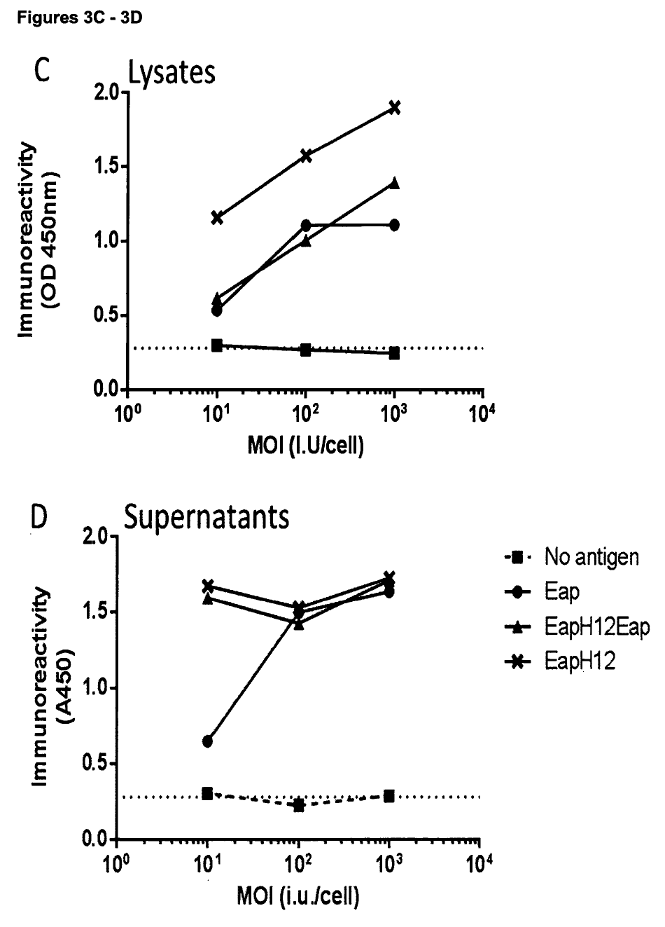

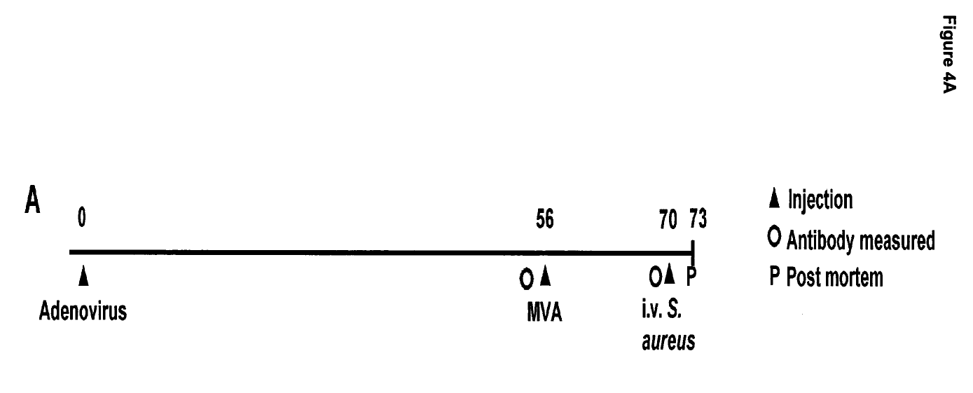

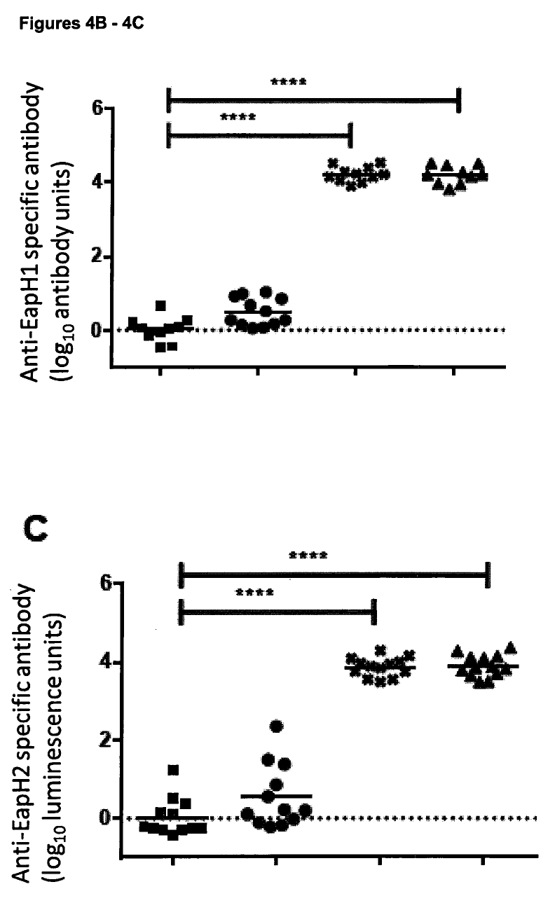

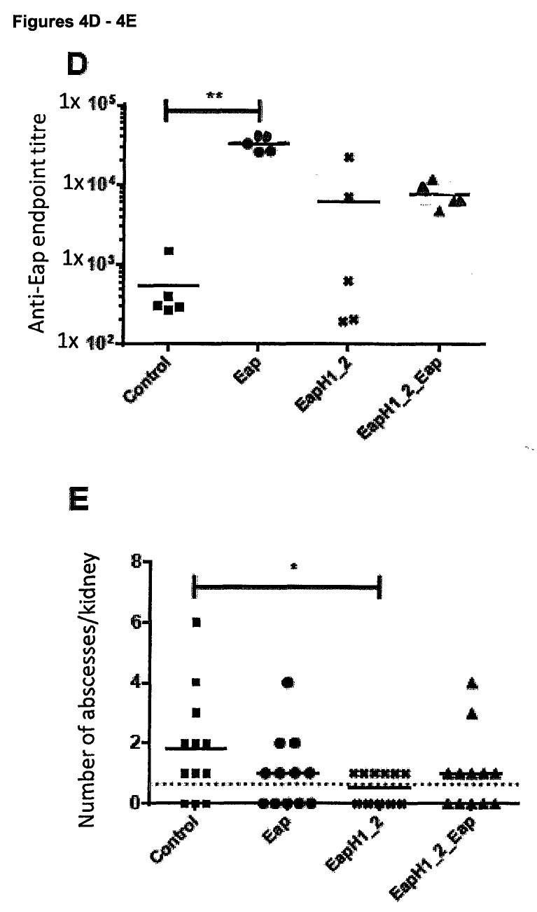

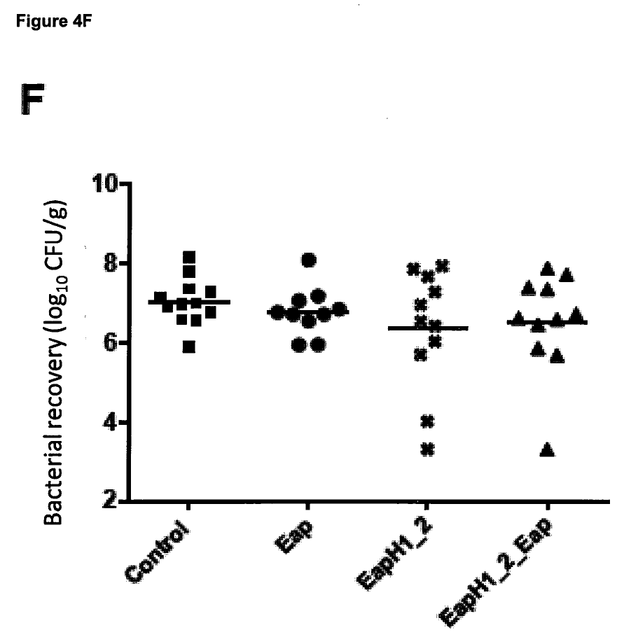

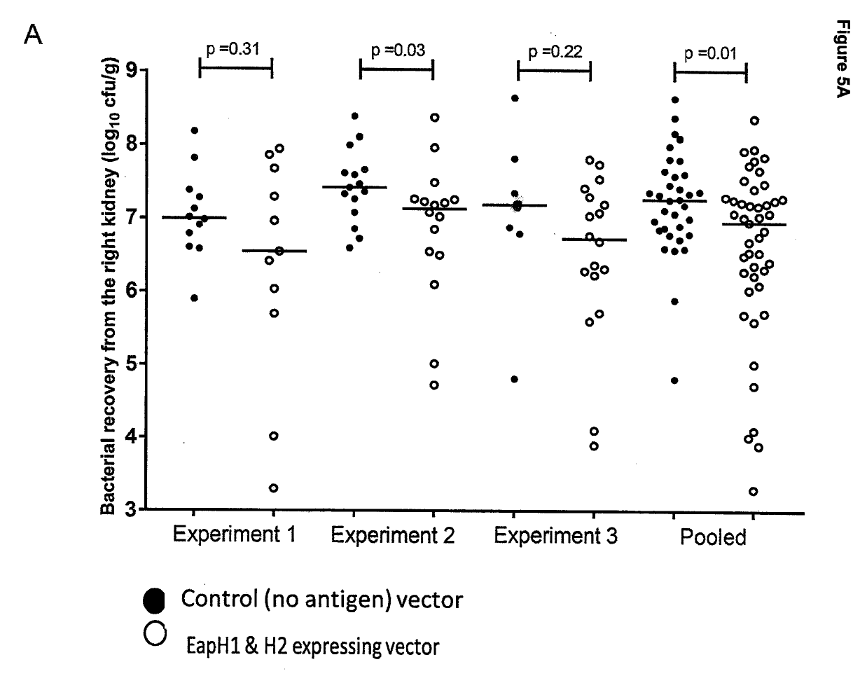

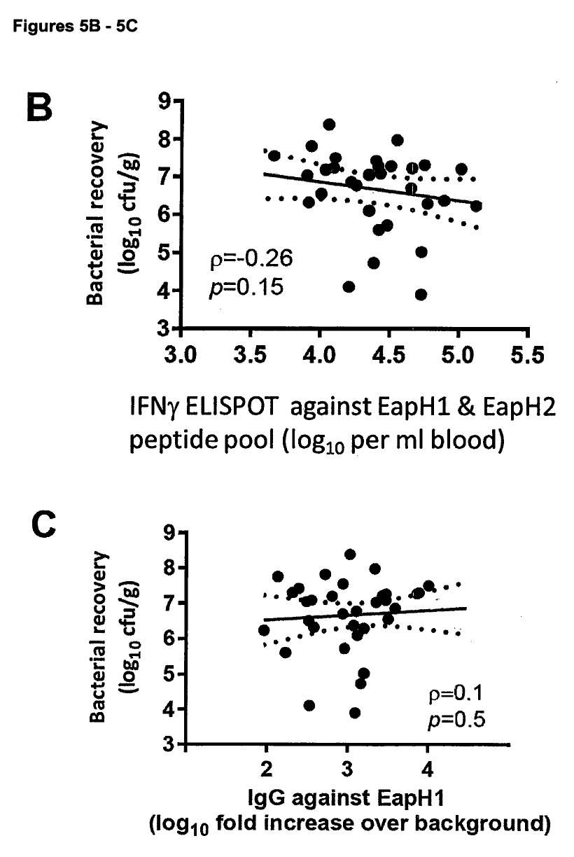

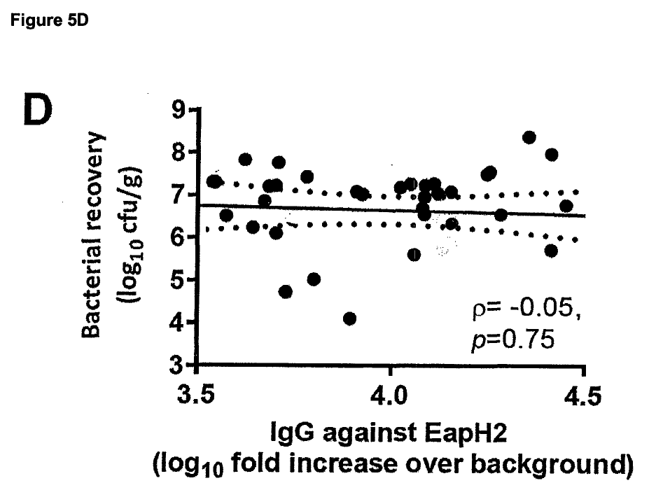

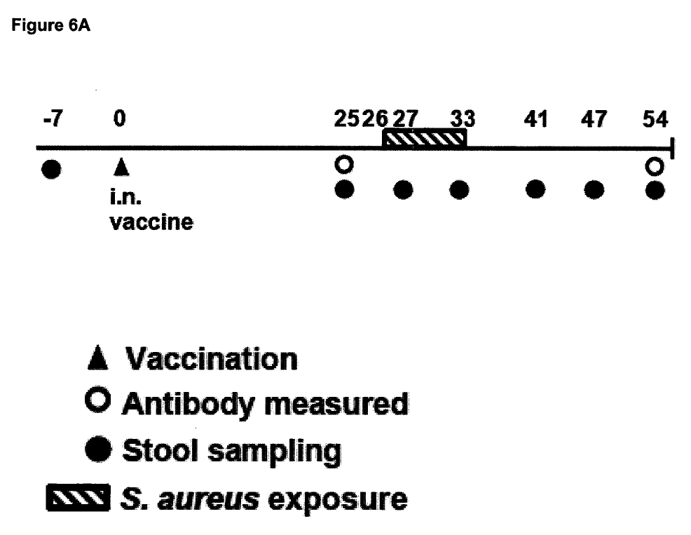

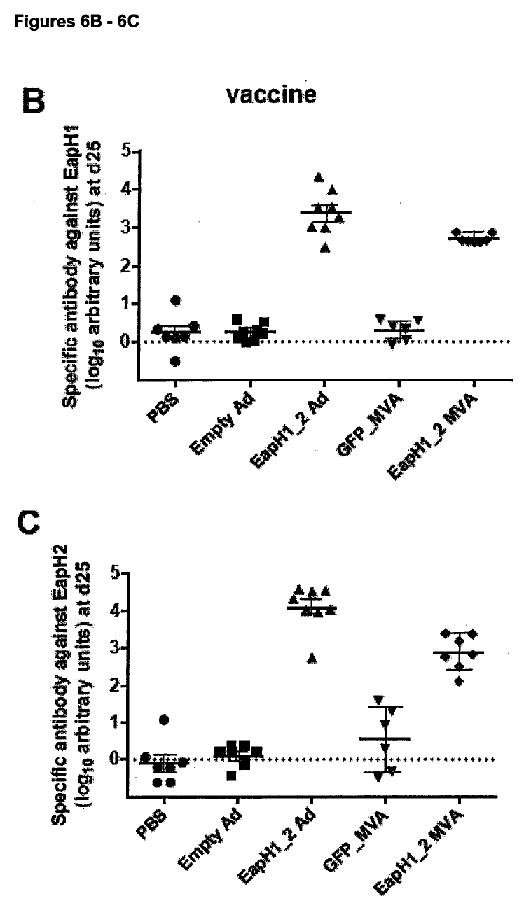

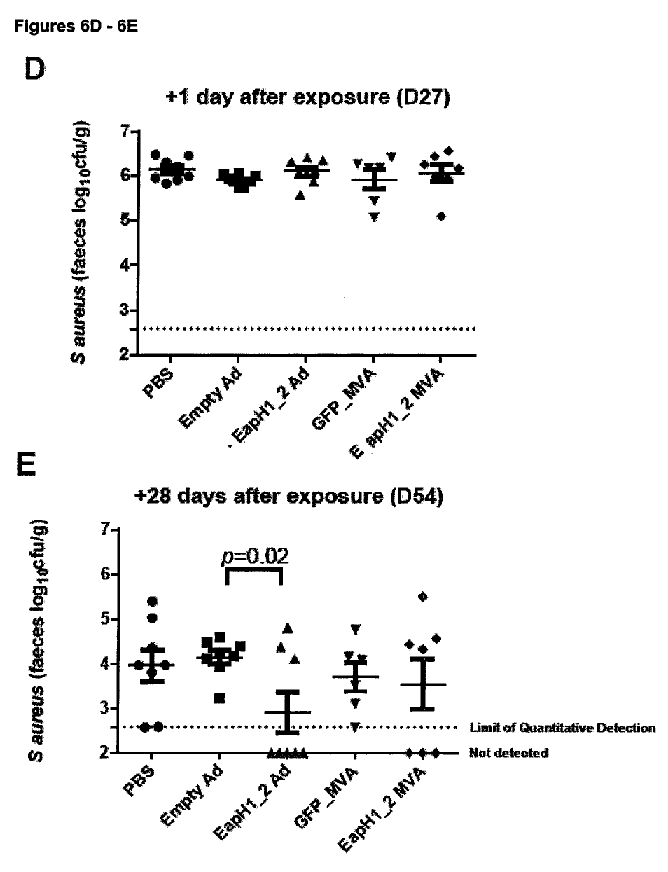

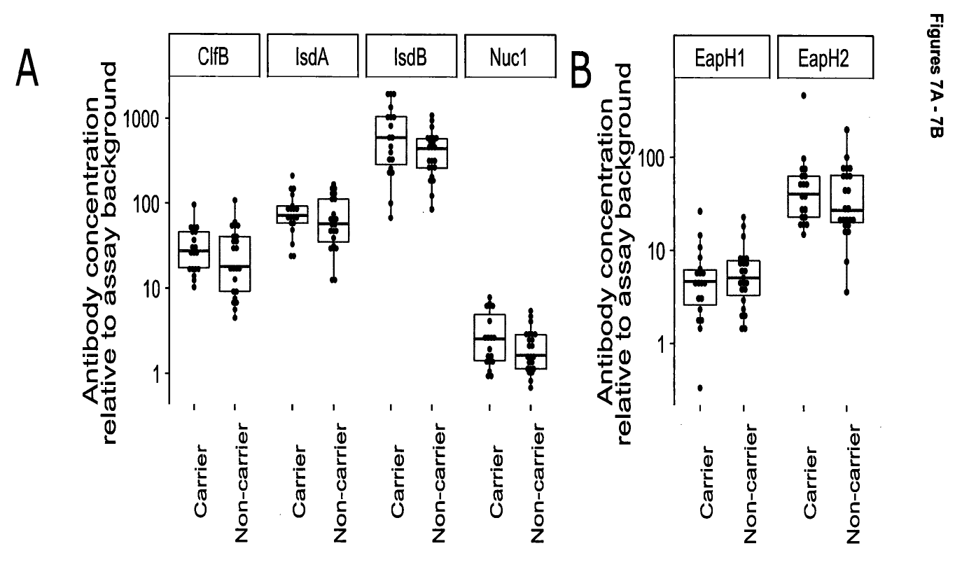

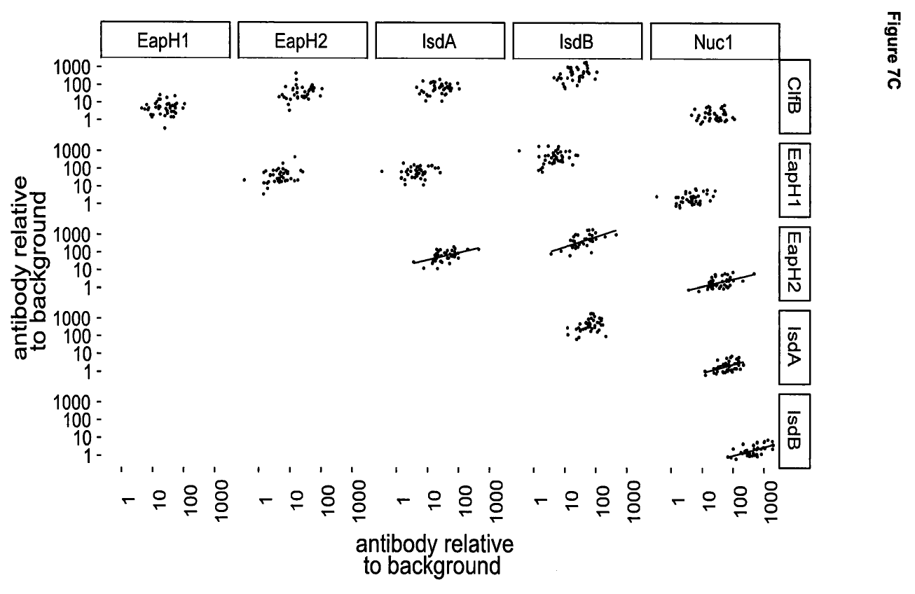

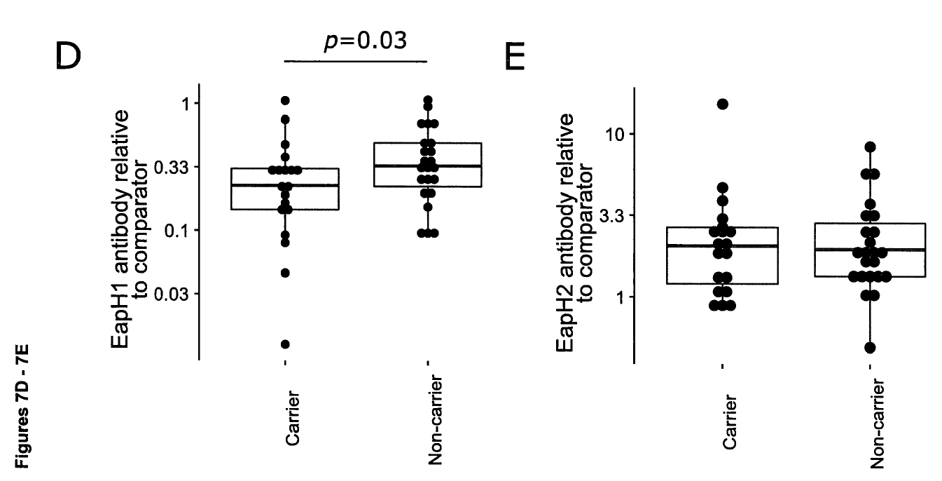

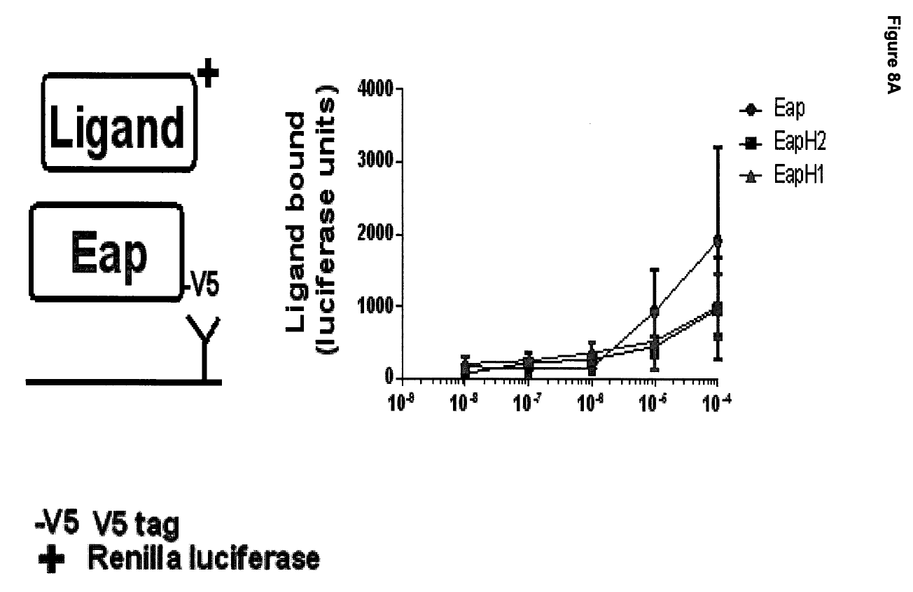

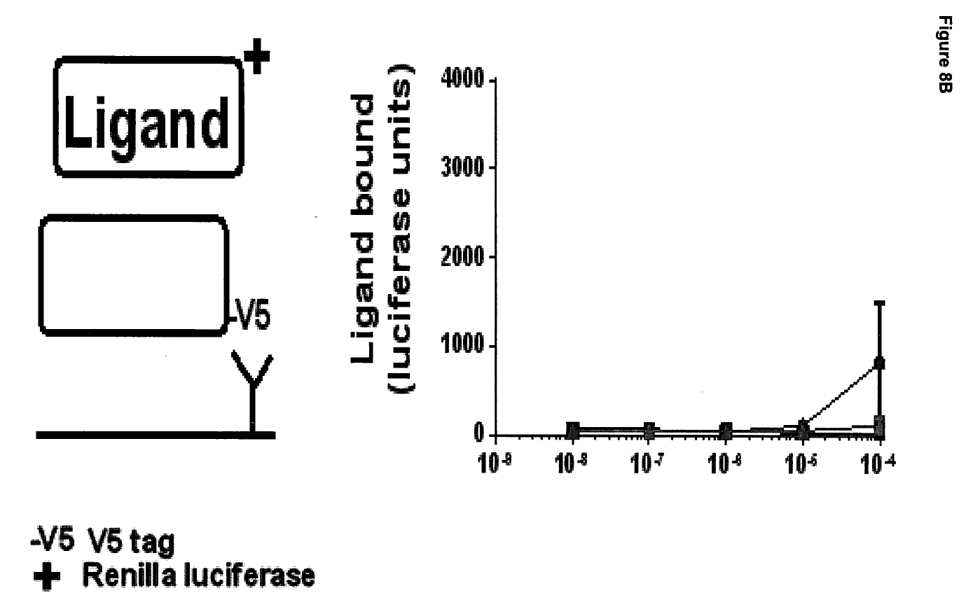

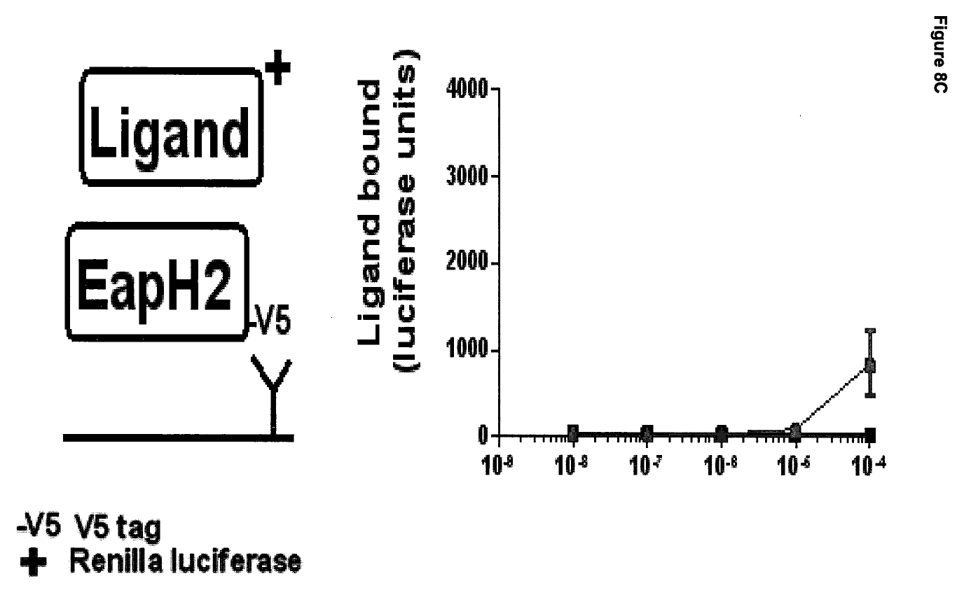

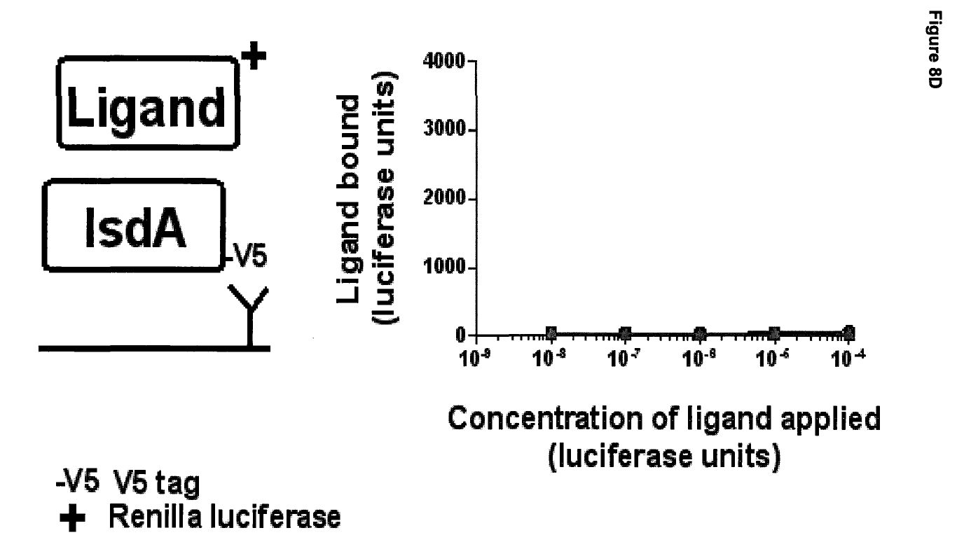

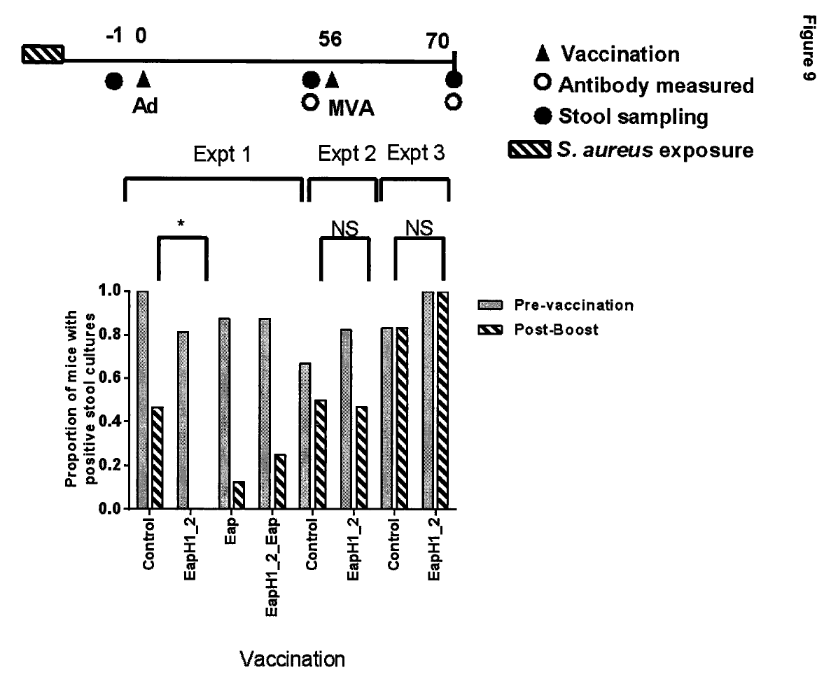

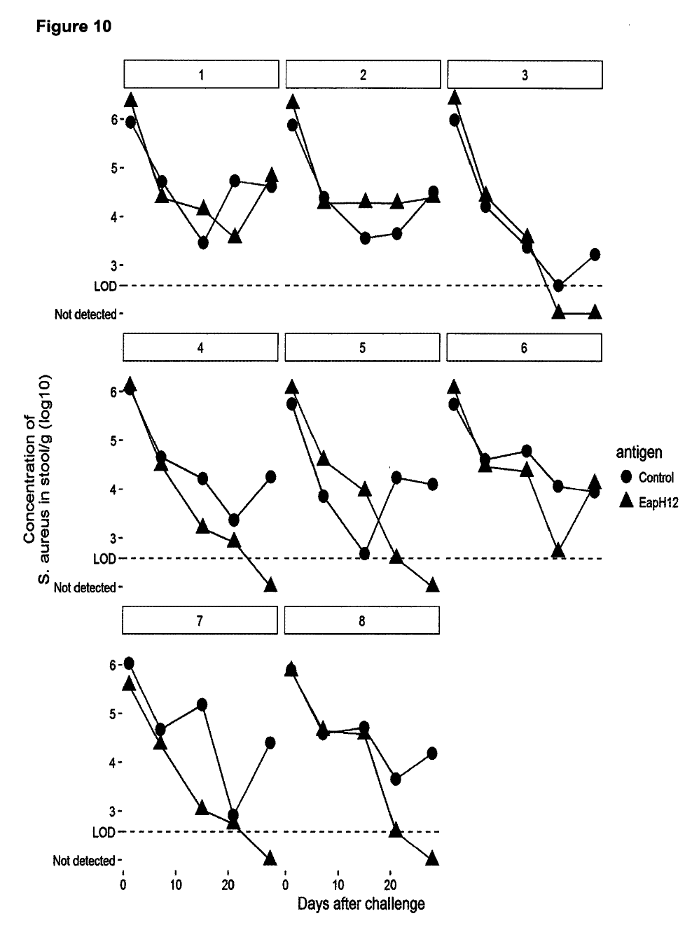

Eap is a multimerising, cell surface associated protein [24]. We purified recombinant His-tagged Eap, EapH1 and EapH2 proteins from E. coli, and removed endotoxin using an affinity column. The His tag was cleaved from an aliquot of each protein, and an interaction assay established in which this material was immobilised on an EIA plate (FIG. 2A). Binding of a His-tagged ligand to the immobilised protein was detected using an anti-His antibody (FIG. 2B-D). We observed apparently saturable binding of Eap to itself with a K_{d} estimate of 80 nM (95% Cl 29, 134), and with EapH2 (estimated K_{d} 82 nM, 95% Cl 30, 132 nM), (FIG. 2 B-D). We also observed binding of EapH2 to EapH1 and to Eap (FIG. 2), but the affinity could not be reliably quantified as we were unable to obtain saturation of binding at the concentrations of protein available. Binding appeared specific, since similar-sized recombinant Staphylococcal cell surface proteins SAUSA300_2132 and SAUSA300_1795 did not show any binding to immobilized Eap, EapH1 or EapH2 proteins (FIG. 2 B-D).

To exclude co-purification of other bacterial proteins or lipopolysaccharides with our recombinant proteins as an explanation for the interactions observed, we expressed Eap, EapH1 and EapH2 in 293 cells with either a C-terminal fusion to either a V5 tag or to Renilla luciferase epitope tags. V5-tagged proteins were captured onto anti-V5 coated plates, and capture of luciferase activity following addition of lysates containing luciferase fusion proteins quantified (FIG. 8). Absolute protein concentrations are not readily determined in this system, and saturation of binding not clearly achieved. Nevertheless, the pattern of binding observed is compatible with the data shown in FIG. 2. Taken together, these data support a model in which EapH1 and EapH2 may associate with each other, with Eap and perhaps other proteins near the cell surface (FIG. 2E). As such, they may be physically co-located, and with overlapping specificities contributing to potent, broad spectrum protease inhibition around the bacterium.

### Example 4: EapH Protein Production from Viral Vectors

In view of this possible co-location of antigens, we investigated combinations of these three proteins as vaccine antigens since they may comprise complementary components of an immunomodulatory complex. Three constructs, designated Eap, EapH12Eap and EapH12 were constructed to contain different combinations of Map domains from the Eap, EapH1 or EapH2 proteins (FIGS. 3A, 3B). The MAP domains were expressed from both Adenovirus and modified Vaccinia Ankara (MVA) vectors as part of an expression cassette containing a eukaryotic leader sequence, and a 3′ V5 tag (for antigen detection) together with an IMX313 multimerising sequence [31], which increases immunogenicity of some proteins. Cytosolic presence and secretion of antigens from viral vectors in vitro was readily detectable using a non-quantitative anti-V5 capture, anti-IMX313 detection sandwich EIA (FIGS. 3C, 3D). Identical adenovirus and MVA vectors, apart from the absence of the antigen expression cassette, were used as controls.

Balb/c mice were immunised with Adenovirus followed by MVA vaccine components (FIG. 4A). As judged by serological analysis on day 70, all components of all the vaccines were immunogenic, producing a strong antibody response against respective Map domains of Eap, EapH1 and EapH2 proteins (FIGS. 4B-D). Antibodies against EapH proteins were assessed by luciferase immunoprecipitation (LIPS) and a subset of sera were analysed by endpoint dilution against recombinant bacterial Eap. Weak immune responses against Eap were observed in 2/5 animals immunised with EapH12 alone, suggesting that some cross reactivity between Eap and EapH1/2 may occur in a subset of animals.

### Example 5: EapH1 and H2 Reduce Abscess Numbers and Bacterial Recovery in i.v. Challenge Models

Following vaccination, mice were challenged i.v. with S. aureus strain Newman. All mice survived the challenge until day 3, when a post mortem was performed. The right kidney of each mouse was analysed using a sensitive post-mortem MRI technique [27], and abscess numbers quantified (FIG. 4E). The number of right kidney abscesses was significantly lower in the EapH12 vaccinated group in comparison to the control group (p=0.04). Bacterial recovery from the left kidney was also used to assess vaccine efficacy. The bacterial recovery in the EapH1H2 vaccine group differed from the control group by 0.7 log₁₀ CFU/g, a difference which is not statistically significant (4F) (p=0.31, Mann-Whitney test).

Since power calculations using historical data from this infection model [32] indicated we are only powered to detect a 0.9 log decrease in bacterial numbers using this experimental design, we performed two additional similar experiments to investigate the impact of EapH1/H2 vaccination on bacterial recovery post i.v. infection. These indicated the effect observed was reproducible and consistent (FIG. 5A). A significant reduction in bacterial recovery was seen following Adenovirus/MVA vaccination with EapH1_2, across the 3 experiments. In the latter two experiments, blood γlFN secreting cells were determined using tail bleeds on day 70, before i.v. challenge. These experiments showed induction of a geometric mean of 24,000 γlFN secreting EapH1/H2 responsive T cells per μl by the Adenovirus/MVA regime. However, neither blood ELISPOT nor serological responses against EapH1 or EapH2 were significantly associated with protection (FIGS. 5B-D). S aureus carriage levels in stools were also monitored and in 1 of the 3 experiments we found that a significantly higher proportion of mice were S. aureus negative in the Adenovirus-EapH1H2 group than the control adenovirus (p=0.006) vaccinated animals (FIG. 9). Thus, the protective mechanism(s) induced by the vaccine are unclear.

### Example 6: Intranasal Vaccination with EapH Proteins Accelerates Loss of S. aureus Carriage

Adherence to mammalian cell surfaces has been proposed as a step in S. aureus carriage [33], as has generation of T cell responses against S. aureus [34]. Since EapH proteins may be exposed on the cell surface, and since viral vectors elicit potent T- and B-cell responses against encoded antigens [35], we tested whether eliciting mucosal responses against these proteins could alter S. aureus colonisation. CD1 mice without S. aureus colonisation were vaccinated intranasally with Adenovirus Hu5 expressing EapH1 and H2, a control Adenovirus expressing no antigen, MVA expressing EapH1 and H2, MVA expressing GFP, or PBS. One mouse from each vaccinated group was placed in each of eight cages (FIG. 6A). Vaccination induced systemic antibody responses against EapH1 and EapH2 (FIG. 6B,C). 26 days later, mice were exposed to S. aureus Newman by environmental contamination. Stool cultures 1 day later were positive in all mice (FIG. 6D). Carriage levels of S. aureus in stools were monitored. In all cages the carriage levels declined over time (FIG. 10), which has been observed previously in the animals in our facility. However, at the end of followup (day 54 after vaccination, 28 days after experimental colonisation), a significantly higher proportion of mice were S. aureus negative in the Adenovirus-EapH1H2 group than the control adenovirus (p=0.02) or PBS vaccinated animals (FIG. 6E). Thus, EapH1H2 vaccination may accelerate loss of carriage in murine models.

### Example 7: Recognition of EapH1 and EapH2 by Human Populations

We examined a cohort of 42 humans, randomly selected from cohort studies, including 19 carriers (identified by having two S. aureus nasal swabs positive) and 23 non-carriers. We quantified IgG antibodies against the cell surface proteins IsdA, IsdB, ClfB and the nuclease Nuc1, antigens selected because antibodies against these proteins are known to be prevalent in humans [37, 38], using an immunoprecipitation assay. Antibodies against all four proteins were detected, with increased concentrations in carriers vs. non-carriers (fold increase 1.32, 95% Cl 1.07-1.64) (FIG. 7A). Antibodies against EapH1 and EapH2 were also detected (FIG. 7B). Significant correlations (Spearman's rho differs from 0, p<0.01) were seen between antibody quantities to Isd proteins and to Nuclease (FIG. 7C), with less significant associations (p<0.05) with ClfB antibodies. Antibody titres against EapH2, but not EapH1, were also associated with anti-Isd and Nuclease responses (FIG. 7C).

In view of this, and our finding that antibody generation against EapH proteins reduced carriage, we considered whether the relationship between antibody against well known S. aureus antigens (IsdA, IsdB, ClfB, Nuc1), and that against EapH proteins, were carriage-specific. Such an association might exist if in individuals with higher anti-EapH protein responses, carriage (and thence exposure to other S. aureus antigens) was inhibited. We computed a mean response to the reference antigens (IsdA, IsdB, ClfB, Nuc1) in each individual and compared with responses to EapH protein. In non-carriers, EapH1 (FIG. 7D), but non-EapH2 (FIG. 7E), responses, were significantly higher relative to the reference antigens. Thus, our data is compatible with the idea that the development of immune responses against EapH1 may be associated with loss of carriage.

SEQUENCES
Complete EapH1 nucleotide sequence from S. aureus Newman
(from WP 001549607.1)
SEQ ID NO: 1
ATGAAACTAAAATCATTTGTTACTGCCACTTTAGCATTGGGATTATTATCAACGGTCGGAGCTG
CATTACCGAGTCACGAAGCATCTGCAGATAGTAATAACGGCTATAAAGAAATGACTGTGGATGG
TTATCACACTGTTCCTTACACAATTTCAGTAGATGGTATTACTGCATTACATCGAACTTACTTT
ATCTTCCCAGAAAATAAAAATGTTCTTTATCAAGAAATTGACAGTAAAGTAAAAAATGAATTAG
CTTCTCAACGTGGTGTTACAACAGAAAAAATTAATAATGCCCAAACAGCAACTTATACGCTTAC
TTTGAATGATGGTAATAAAAAAGTAGTGAATCTAAAGAAAAATGACGACGCTAAAAATTCAATT
GATCCAAGTACAATCAAACAGATACAAATTGTAGTTAAATAA
Complete EapH1 amino acid sequence from S. aureus Newman
(from WP 001549607.1)
SEQ ID NO: 2
MKLKSFVTATLALGLLSTVGAALPSHEASADSNNGYKEMTVDGYHTVPYTISVDGITALHRTYF
IFPENKNVLYQEIDSKVKNELASQRGVTTEKINNAQTATYTLTLNDGNKKVVNLKKNDDAKNSI
DPSTIKQIQIVVK
Codon optimised EapH1 antigen nucleotide sequence from S. aureus
Newman
SEQ ID NO: 3
CACGAGGCCTCTGCCGATAGCAACAACGGCTACAAAGAAATGACCGTGGATGGCTACCACACCG
TGCCTTACACCATCTCTGTGGATGGAATCACCGCCCTGCACCGGACCTACTTCATCTTCCCCGA
GAACAAGAACGTGCTGTACCAGGAAATCGACTCTAAAGTGAAGAACGAGCTGGCCTCCCAGAGA
GGCGTGACAACCGAGAAGATTAACAACGCCCAGACCGCCACCTACACCCTGACCCTGAACGACG
GCAACAAAAAGGTCGTGAATCTGAAGAAGAACGACGACGCCAAGAACAGCATCGACCCCAGCAC
CATTAAGCAGATCCAGATCGTCGTGAAG
EapH1 antigen amino acid sequence from S. aureus Newman
SEQ ID NO: 4
HEASADSNNGYKEMTVDGYHTVPYTISVDGITALHRTYFIFPENKNVLY
QEIDSKVKNELASQRGVTTEKINNAQTATYTLTLNDGNKKVVNLKKNDDA
KNSIDPSTIKQIQIVVK
Complete EapH2 nucleotide sequence from S. aureus Newman
(from WP 000769689.1)
SEQ ID NO: 5
ATGAAATTAAAATCATTTATAACTGTAACTTTGGCACTGGGCATGATCGCAACGACTGGCGCTA
CTGTGGCAGGTAATGAGGTATCTGCAGCAGAAAAGGACAAACTACCGGCAACTCAAAAAGCTAA
AGAAATGCAAAATGTTCCATATACAATTGCAGTAGATGGCATTATGGCTTTCAATCAATCTTAC
TTAAATTTACCAAAAGATAGCCAATTATCATATTTAGATTTAGGAAATAAAGTTAAAGCTTTGT
TATATGATGAACGCGGTGTAACACCTGAGAAGATTCGAAATGCAAAATCTGCCGTTTACACGAT
TACTTGGAAAGATGGTAGTAAAAAAGAAGTGGATCTTAAGAAAGATAGCTACACAGCAAACTTG
TTTGATTCAAATTCAATTAAACAAATTGATATTAATGTAAAAACTAAATAA
Complete EapH2 amino acid sequence from S. aureus Newman
(from WP 000769689.1)
SEQ ID NO: 6
MKLKSFITVTLALGMIATTGATVAGNEVSAAEKDKLPATQKAKEMQNVPYTIAVDGIMAFNQSY
LNLPKDSQLSYLDLGNKVKALLYDERGVTPEKIRNAKSAVYTITWKDGSKKEVDLKKDSYTANL
FDSNSIKQIDINVKTK
Codon optimised EapH2 antigen nucleotide sequence from S. aureus
Newman
SEQ ID NO: 7
AACGAGGTGTCAGCCGCCGAGAAGGATAAGCTGCCCGCCACCCAGAAAGCCAAAGAAATGCAGA
ACGTGCCCTACACAATCGCCGTGGACGGCATCATGGCCTTCAACCAGAGCTACCTGAACCTGCC
CAAGGACAGCCAGCTGAGCTATCTGGACCTGGGCAACAAAGTGAAGGCCCTGCTGTACGACGAG
CGGGGCGTGACCCCTGAGAAGATCAGAAACGCCAAGAGCGCCGTGTACACCATCACCTGGAAGG
ACGGCAGCAAGAAAGAGGTGGACCTGAAGAAGGACAGCTACACCGCCAACCTGTTCGACAGCAA
CAGCATCAAGCAGATCGACATCAACGTGAAAACAAAG
EapH2 MAP domain amino acid sequence from S. aureus Newman
SEQ ID NO: 8
NEVSAAEKDKLPATQKAKEMQNVPYTIAVDGIMAFNQSYLNLPKDSQL
SYLDLGNKVKALLYDERGVTPEKIRNAKSAVYTITWKDGSKKEVDLKKDSYT
ANLFDSNSIKQIDINVKTK
Eap MAP domain amino acid sequence from S. aureus Newman
(from WP_001549158.1)
SEQ ID NO: 9
VPYTIAVNGTSTPILSKLKISNKQLISYKYLNDKVKSVLKSERGISDLDLKFAK
QAKYTVYFKNGKKQVVNLKSDIFTPNLFSAKDIKKIDIDVKQYTKSKKNK
Codon-optimised Eap MAP nucleotide sequence
SEQ ID NO: 10
GTTCCGGAGTGCCCTATACCATTGCTGTGAACGGCACCAGCACCCCCATCCTGAGCAAGCTGAAGATCAG
CAACAAGCAGCTGATCTCCTACAAGTACCTGAACGACAAAGTGAAAAGCGTGCTGAAGTCCGAGAGAGGC
ATCAGCGACCTGGATCTGAAGTTCGCCAAGCAGGCCAAGTACACCGTGTATTTCAAGAACGGGAAGAAAC
AGGTCGTGAACCTGAAAAGCGACATCTTCACCCCTAATCTGTTCAGCGCCAAGGACATCAAGAAAATTGA
TATCGACGTGAAGCAGTACACCAAGAGCAAGAAGAACAAGGGATCCGGGCCCGGG
Eap protein amino acid sequences from Strain Newman
(from NCBI locus CAB94853.1)
SEQ ID NO: 11
MKFKSLITTTLALGVLASTGANFNNNEASAAAKPLDKSSSSLHHGYSKVHVPYAITVNGTSQNILSSLTF
NKNQNISYKDLEDRVKSVLKSDRGISDIDLRLSKQAKYTVYFKNGTKKVIDLKVGIYTADLINTSEIKAI
NINVDTKKQVEDKKKDKANYQVPYTITVNGTSQNILSNLTFNKNQNISYKDLEDKVKSVLESNRGITDVD
LRLSKQAKYTVNFKNGTKKVIDLKSGIYTANLINSSDIKSININVDTKKHIENKAKRNYQVPYSINLNGT
STNILSNLSFSNKPWTNYKNLTSQIKSVLKHDRGISEQDLKYAKKAYYTVYFKNGGKRILQLNSKNYTAN
LVHAKDVKRIEITVKTGTKAKADRYVPYTIAVNGTSTPILSDLKFTGDPRVGYKDISKKVKSVLKHDRGI
GERELKYAKKATYAVHFKNGTKKVININSNISQLNLLYVQDIKKIDIDVKTGTKAKADSYVPYTIAVNGT
STPILSKLKISNKQLISYKYLNDKVKSVLKSERGISDLDLKFAKQAKYTVYFRNGKKQVVNLKSDIFTPN
LFSAKDIKKIGIDVKQYTKSKKNK
Staphylococcus aureus map-ND2C gene (AJ290973.2)
SEQ ID NO: 12
AATTATCATAAAAAAGGAGTGATAATTTATGAAATTTAAGTCATTGATTACAACAACTTTAGCATTAGGT
GTTTTAGCATCAACAGGTGCAAACTTTAATAATAATGAAGCGTCTGCCGCAGCTAAGCCATTAGATAAAT
CATCAAGTTCGTTACACCATGGATATTCTAAAGTCCATGTTCCATATGCAATCACTGTGAACGGTACAAG
CCAAAATATTTTATCAAGCTTAACATTTAATAAGAATCAAAATATTAGTTATAAAGATTTAGAGGATAGA
GTTAAATCAGTTTTAAAATCAGACAGAGGTATTAGTGATATAGATTTAAGACTATCGAAGCAAGCGAAAT
ATACTGTTTACTTTAAAAATGGAACAAAGAAAGTTATCGATTTGAAAGTAGGTATTTACACAGCTGATTT
AATTAATACAAGTGAAATTAAAGCTATTAATATTAACGTAGATACTAAAAAGCAAGTTGAAGATAAAAAG
AAAGATAAAGCAAATTACCAAGTTCCATACACAATCACTGTGAACGGTACAAGCCAAAATATTTTATCAA
ACTTAACATTTAATAAGAATCAAAATATTAGTTACAAAGATTTAGAGGATAAAGTTAAATCAGTTTTAGA
ATCAAATAGAGGTATTACCGATGTTGATTTAAGATTATCGAAGCAAGCGAAATATACAGTTAATTTTAAA
AATGGAACGAAGAAAGTTATCGATTTGAAATCAGGTATTTACACAGCGAATTTAATAAATTCAAGTGATA
TTAAAAGTATCAATATTAACGTAGATACAAAAAAACATATCGAAAATAAAGCTAAAAGAAACTATCAAGT
TCCATATTCAATTAATTTAAATGGTACATCTACAAACATTTTATCGAATCTTTCATTTTCAAATAAACCT
TGGACAAATTACAAAAATTTAACTAGTCAAATAAAATCAGTACTGAAGCATGATAGAGGTATTAGTGAAC
AAGATTTAAAATATGCTAAGAAAGCTTATTATACTGTTTATTTTAAAAATGGTGGTAAAAGAATCTTACA
GTTGAATTCAAAAAATTACACAGCAAACTTAGTTCATGCGAAAGATGTTAAGAGAATTGAAATTACTGTT
AAAACAGGAACTAAAGCGAAAGCAGACAGATATGTACCATACACAATTGCAGTAAATGGCACATCAACAC
CAATTTTATCAGATTTAAAATTTACAGGTGACCCACGTGTAGGCTACAAAGATATCTCTAAAAAAGTTAA
ATCAGTACTGAAGCATGATAGAGGTATCGGGGAACGTGAATTAAAATATGCAAAAAAAGCTACTTACGCA
GTACATTTTAAAAATGGAACGAAAAAAGTGATTAACATAAATTCAAATATTAGCCAACTGAATCTGCTTT
ATGTTCAAGATATTAAAAAGATAGATATTGATGTTAAAACAGGAACTAAAGCGAAAGCGGATAGCTATGT
ACCATATACAATTGCAGTAAATGGCACATCAACACCAATTTTATCAAAACTTAAAATTTCGAATAAACAA
TTAATTAGTTACAAATATTTAAACGACAAAGTGAAATCTGTATTAAAAAGTGAAAGAGGCATCAGTGATC
TTGACTTAAAATTTGCGAAACAAGCAAAATATACAGTATATTTCAGAAATGGAAAGAAACAAGTAGTGAA
TTTAAAATCAGACATCTTTACACCTAATTTATTTAGTGCCAAAGATATTAAAAAGATTGGTATTGATGTA
AAACAATACACTAAATCAAAAAAAAATAAATAAATTCTAATAATGTGAAATTCCCAGTAACAATAAATAA
ATTTGAAAACATAGTTTCAAATGAATTTGTGTTCTATAATGCAAGCAAAATTACAATTAATGATTTAAGT
ATAAAACTTAAATCAGCAATGGCAAATGATCAAGGGATAACTAAACATGACATAGGACTTGCTGAACGCG
CAGTGTATAAAGTGTATTTTAAAAATGGTTCGTCAAAATATGTAGACTTAAAAACTGAGTATAAAGATGA
AAGAGTATTTAAAGCAACTGACATTAAAAAAGTAGAAATTGAAATTAAATTTTAA
TPA leader:
SEQ ID NO: 13
ATGAAGAGAGGGCTCTGCTGTGTGCTGCTGCTGTGTGGAGCAGTCTTCGTTTCGCCCAGCCAGG
AAATCCATGCCCGATTCAGAAGA
BGH polyA:
SEQ ID NO: 14
ctgtgccttctagttgccagccatctgttgtttgcccctcccccgtgccttccttgaccctgga
aggtgccactcccactgtcctttcctaataaaatgaggaaattgcatcg
Cattgtctgagtaggtgtcattctattctggggggtggggtggggcaggacagcaagggggagg
attgggaagacaatagcaggcatgctggggatgcggtgggctctatgg

### REFERENCES

1. Lowy F D. Staphylococcus aureus infections. The New England journal of medicine. 1998; 339(8):520-32. doi: 10.1056/NEJM199808203390806. PubMed PMID: 9709046.
2. French G L. The continuing crisis in antibiotic resistance. International journal of antimicrobial agents. 2010; 36 Suppl 3:S3-7. doi: 10.1016/S0924-8579(10)70003-0. PubMed PMID: 21129629.
3. Fowler V G, Jr., Proctor R A. Where does a Staphylococcus aureus vaccine stand?Clinical microbiology and infection: the official publication of the European Society of Clinical Microbiology and Infectious Diseases. 2014; 20 Suppl 5:66-75. Epub 2014/01/31. doi: 10.1111/1469-0691.12570. PubMed PMID: 24476315; PubMed Central PMCID: PMCPMC4067250.
4. von Eiff C, Becker K, Machka K, Stammer H, Peters G. Nasal carriage as a source of Staphylococcus aureus bacteremia. Study Group. The New England journal of medicine. 2001; 344(1): 11-6. Epub 2001/01/04. doi: 10.1056/nejm200101043440102. PubMed PMID: 11136954.
5. Huang S S, Septimus E, Kleinman K, Moody J, Hickok J, Avery T R, et al. Targeted versus universal decolonization to prevent ICU infection. The New England journal of medicine. 2013; 368(24):2255-65. Epub 2013/05/31. doi: 10.1056/NEJMoa1207290. PubMed PMID: 23718152.
6. Bode L G, Kluytmans J A, Wertheim H F, Bogaers D, Vandenbroucke-Grauls C M, Roosendaal R, et al. Preventing surgical-site infections in nasal carriers of Staphylococcus aureus. The New England journal of medicine. 2010; 362(1):9-17. Epub 2010/01/08. doi: 10.1056/NEJMoa0808939. PubMed PMID: 20054045.
7. Bode L G, van Rijen M M, Wertheim H F, Vandenbroucke-Grauls C M, Troelstra A, Voss A, et al. Long-term Mortality After Rapid Screening and Decolonization of Staphylococcus Aureus Carriers: Observational Follow-up Study of a Randomized, Placebo-controlled Trial. Annals of surgery. 2016; 263(3):511-5. Epub 2015/11/14. doi: 10.1097/sla.0000000000001060. PubMed PMID: 26565136.
8. David M Z, Siegel J D, Henderson J, Leos G, Lo K, Iwuora J, et al. A randomized, controlled trial of chlorhexidine-soaked cloths to reduce methicillin-resistant and methicillin-susceptible Staphylococcus aureus carriage prevalence in an urban jail. Infection control and hospital epidemiology. 2014; 35(12):1466-73. Epub 2014/11/25. doi: 10.1086/678606. PubMed PMID: 25419768.
9. Derde L P, Cooper B S, Goossens H, Malhotra-Kumar S, Willems R J, Gniadkowski M, et al. Interventions to reduce colonisation and transmission of antimicrobial-resistant bacteria in intensive care units: an interrupted time series study and cluster randomised trial. Lancet Infect Dis. 2014; 14(1):31-9. Epub 2013/10/29. doi: 10.1016/s1473-3099(13)70295-0. PubMed PMID: 24161233; PubMed Central PMCID: PMCPMC3895323.
10. Whitman T J, Schlett C D, Grandits G A, Millar E V, Mende K, Hospenthal D R, et al. Chlorhexidine gluconate reduces transmission of methicillin-resistant Staphylococcus aureus USA300 among Marine recruits. Infection control and hospital epidemiology. 2012; 33(8):809-16. Epub 2012/07/05. doi: 10.1086/666631. PubMed PMID: 22759549.
11. Cheng A G, DeDent A C, Schneewind O, Missiakas D. A play in four acts: Staphylococcus aureus abscess formation. Trends in microbiology. 2011; 19(5):225-32. Epub 2011/03/01. doi: 10.1016/j.tim.2011.01.007. PubMed PMID: 21353779; PubMed Central PMCID: PMCPMC3087859.
12. Lehar S M, Pillow T, Xu M, Staben L, Kajihara K K, Vandlen R, et al. Novel antibody-antibiotic conjugate eliminates intracellular S. aureus. Nature. 2015; 527(7578):323-8. Epub 2015/11/05. doi: 10.1038/nature16057. PubMed PMID: 26536114.
13. McGuinness W A, Kobayashi S D, DeLeo F R. Evasion of Neutrophil Killing by Staphylococcus aureus. Pathogens. 2016; 5(1). Epub 2016/03/22. doi: 10.3390/pathogens5010032. PubMed PMID: 26999220; PubMed Central PMCID: PMCPMC4810153.
14. Das S, Lindemann C, Young B C, Muller J, Osterreich B, Ternette N, et al. Natural mutations in a Staphylococcus aureus virulence regulator attenuate cytotoxicity but permit bacteremia and abscess formation. Proc Natl Acad Sci USA. 2016; 113(22): E3101-10. Epub 2016/05/18. doi: 10.1073/pnas. 1520255113. PubMed PMID: 27185949; PubMed Central PMCID: PMCPMC4896717.
15. Thammavongsa V, Kim H K, Missiakas D, Schneewind O. Staphylococcal manipulation of host immune responses. Nat Rev Microbiol. 2015; 13(9):529-43. doi: 10.1038/nrmicro3521. PubMed PMID: 26272408; PubMed Central PMCID: PMCPMC4625792.
16. Bagnoli F, Fontana M R, Soldaini E, Mishra R P, Fiaschi L, Cartocci E, et al. Vaccine composition formulated with a novel TLR7-dependent adjuvant induces high and broad protection against Staphylococcus aureus. Proc Natl Acad Sci USA. 2015; 112(12):3680-5. Epub 2015/03/17. doi: 10.1073/pnas.1424924112. PubMed PMID: 25775551; PubMed Central PMCID: PMCPMC4378396.
17. Brown A F, Murphy A G, Lalor S J, Leech J M, O'Keeffe K M, Mac Aogain M, et al. Memory Th1 Cells Are Protective in Invasive Staphylococcus aureus Infection. PLoS Pathog. 2015; 11(11):e1005226. Epub 2015/11/06. doi: 10.1371/journal.ppat.1005226. PubMed PMID: 26539822; PubMed Central PMCID: PMCPMC4634925.
18. Garzoni C, Francois P, Huyghe A, Couzinet S, Tapparel C, Charbonnier Y, et al. A global view of Staphylococcus aureus whole genome expression upon internalization in human epithelial cells. BMC Genomics. 2007; 8:171. Epub 2007/06/16. doi: 1471-2164-8-171 [pii]10.1186/1471-2164-8-171. PubMed PMID: 17570841; PubMed Central PMCID: PMC1924023.
19. Stapels D A, Kuipers A, von Kockritz-Blickwede M, Ruyken M, Tromp A, Horsburgh M J, et al. Staphylococcus aureus protects its immune-evasion proteins against degradation by neutrophil serine proteases. Cellular microbiology. 2015. doi: 10.1111/cmi.12528. PubMed PMID: 26418545.
20. Stapels D A, Ramyar K X, Bischoff M, von Kockritz-Blickwede M, Milder F J, Ruyken M, et al. Staphylococcus aureus secretes a unique class of neutrophil serine protease inhibitors. Proc Natl Acad Sci USA. 2014; 111(36):13187-92. doi: 10.1073/pnas.1407616111. PubMed PMID: 25161283; PubMed Central PMCID: PMC4246989.
21. Hammel M, Nemecek D, Keightley J A, Thomas G J, Jr., Geisbrecht B V. The Staphylococcus aureus extracellular adherence protein (Eap) adopts an elongated but structured conformation in solution. Protein science: a publication of the Protein Society. 2007; 16(12):2605-17. doi: 10.1110/ps.073170807. PubMed PMID: 18029416; PubMed Central PMCID: PMC2222813.
22. Cheng A G, Kim H K, Burts M L, Krausz T, Schneewind O, Missiakas D M. Genetic requirements for Staphylococcus aureus abscess formation and persistence in host tissues. FASEB journal: official publication of the Federation of American Societies for Experimental Biology. 2009; 23(10):3393-404. doi: 10.1096/fj.09-135467. PubMed PMID: 19525403; PubMed Central PMCID: PMC2747682.
23. Chavakis T, Hussain M, Kanse S M, Peters G, Bretzel R G, Flock J I, et al. Staphylococcus aureus extracellular adherence protein serves as anti-inflammatory factor by inhibiting the recruitment of host leukocytes. Nature medicine. 2002; 8(7):687-93. doi: 10.1038/nm728. PubMed PMID: 12091905.
24. Palma M, Haggar A, Flock J I. Adherence of Staphylococcus aureus is enhanced by an endogenous secreted protein with broad binding activity. Journal of bacteriology. 1999; 181(9):2840-5. PubMed PMID: 10217776; PubMed Central PMCID: PMC93727.
25. Woehl J L, Stapels D A, Garcia B L, Ramyar K X, Keightley A, Ruyken M, et al. The extracellular adherence protein from Staphylococcus aureus inhibits the classical and lectin pathways of complement by blocking formation of the C3 proconvertase. Journal of immunology. 2014; 193(12):6161-71. doi: 10.4049/jimmunol.1401600. PubMed PMID: 25381436; PubMed Central PMCID: PMC4258549.
26. Everitt R G, Didelot X, Batty E M, Miller R R, Knox K, Young B C, et al. Mobile elements drive recombination hotspots in the core genome of Staphylococcus aureus. Nat Commun. 2014; 5:3956. doi: 10.1038/ncomms4956. PubMed PMID: 24853639; PubMed Central PMCID: PMCPMC4036114.
27. Allen E R, Diemen Pv, Yamaguchi Y, Lindemann C, Thornton V, Soilleux E, et al. MRI based quantification of abscesses following experimental S. aureus intravenous challenge: application to vaccine evaluation.
28. Spencer A J, Hill F, Honeycutt J D, Cottingham M G, Bregu M, Rollier C S, et al. Fusion of the Mycobacterium tuberculosis antigen 85A to an oligomerization domain enhances its immunogenicity in both mice and non-human primates. PLoS One. 2012; 7(3):e33555. doi: 10.1371/journal.pone.0033555. PubMed PMID: 22470455; PubMed Central PMCID: PMC3314664.
29. Whitehouse J, Flaxman A, Rollier C, O'Shea M K, Fallowfield J, Lindsay M, et al. Population variation in anti-S. aureus IgG isotypes influences surface protein A mediated immune subversion. Vaccine. 2016; 34(15):1792-9. doi: 10.1016/j.vaccine.2016.02.034. PubMed PMID: 26921780.
30. Flaxman A, Allen E, Lindemann C, Yamaguchi Y, O'Shea M K, Fallowfield J L, et al. Risk factors for dermatitis in submariners during a submerged patrol: an observational cohort study. BMJ open. 2016; 6(6):e010975. Epub 2016/06/04. doi: 10.1136/bmjopen-2015-010975. PubMed PMID: 27256090; PubMed Central PMCID: PMCPMC4893864.
31. Li Y, Leneghan D B, Miura K, Nikolaeva D, Brian I J, Dicks M D, et al. Enhancing immunogenicity and transmission-blocking activity of malaria vaccines by fusing Pfs25 to IMX313 multimerization technology. Sci Rep. 2016; 6:18848. Epub 2016/01/09. doi: 10.1038/srep18848. PubMed PMID: 26743316; PubMed Central PMCID: PMCPMC4705524.
32. van Diemen P M, Yamaguchi Y, Paterson G K, Rollier C S, Hill A V, Wyllie D H. Irradiated wild-type and Spa mutant Staphylococcus aureus induce anti-S. aureus immune responses in mice which do not protect against subsequent intravenous challenge. Pathog Dis. 2013; 68(1):20-6. Epub 2013/04/27. doi: 10.1111/2049-632x. 12042. PubMed PMID: 23620394.
33. Johannessen M, Sollid J E, Hanssen A M. Host- and microbe determinants that may influence the success of S. aureus colonization. Frontiers in cellular and infection microbiology. 2012; 2:56. Epub 2012/08/25. doi: 10.3389/fcimb.2012.00056. PubMed PMID: 22919647; PubMed Central PMCID: PMCPMC3417514.
34. Archer N K, Harro J M, Shirtliff M E. Clearance of Staphylococcus aureus nasal carriage is T cell dependent and mediated through interleukin-17A expression and neutrophil influx. Infect Immun. 2013; 81(6):2070-5. Epub 2013/03/27. doi: 10.1128/iai.00084-13. PubMed PMID: 23529621; PubMed Central PMCID: PMCPMC3676016.
35. Ewer K J, Lambe T, Rollier C S, Spencer A J, Hill A V, Dorrell L. Viral vectors as vaccine platforms: from immunogenicity to impact. Current opinion in immunology. 2016; 41:47-54. Epub 2016/06/11. doi: 10.1016/j.coi.2016.05.014. PubMed PMID: 27286566.
36. Amy Flaxman PMvD, Yuko Yamaguchi, Christine Rollier, EIizabeth Allen, Claudia Lindemann & David H. Wyllie. An Assessment of Naturally Acquired Immunity and Vaccine Efficacy in a Murine Model of Long Term S. aureus Colonisation.
37. Verkaik N J, Lebon A, de Vogel C P, Hooijkaas H, Verbrugh H A, Jaddoe V W, et al. Induction of antibodies by Staphylococcus aureus nasal colonization in young children. Clinical microbiology and infection: the official publication of the European Society of Clinical Microbiology and Infectious Diseases. 2010; 16(8):1312-7. Epub 2009/10/17. doi: 10.1111/j.1469-0691.2009.03073.x. PubMed PMID: 19832714.
38. Verbrugh H A, Nelson R D, Peterson P K, Wilkinson B J, Thompson R L. Serology of Staphylococcus aureus infections using multiple antigens and serial serum samples. The Journal of infectious diseases. 1983; 148(3):608. Epub 1983/09/01. PubMed PMID: 6619582.
39. Liu C M, Price L B, Hungate B A, Abraham A G, Larsen L A, Christensen K, et al. Staphylococcus aureus and the ecology of the nasal microbiome. Science advances. 2015; 1(5):e1400216. Epub 2015/11/26. doi: 10.1126/sciadv.1400216. PubMed PMID: 26601194; PubMed Central PMCID: PMCPMC4640600.
40. Stranger-Jones Y K, Bae T, Schneewind O. Vaccine assembly from surface proteins of Staphylococcus aureus. Proc Natl Acad Sci USA. 2006; 103(45):16942-7. Epub 2006/11/01. doi: 10.1073/pnas.0606863103. PubMed PMID: 17075065; PubMed Central PMCID: PMCPMC1636558.
41. Wertheim H F, Walsh E, Choudhurry R, Melles D C, Boelens H A, Miajlovic H, et al. Key role for clumping factor B in Staphylococcus aureus nasal colonization of humans. PLoS medicine. 2008; 5(1):el7. Epub 2008/01/18. doi: 10.1371/journal.pmed.0050017. PubMed PMID: 18198942; PubMed Central PMCID: PMCPMC2194749.
42. Harraghy N, Hussain M, Haggar A, Chavakis T, Sinha B, Herrmann M, et al. The adhesive and immunomodulating properties of the multifunctional Staphylococcus aureus protein Eap. Microbiology. 2003; 149(Pt 10):2701-7. doi: 10.1099/mic.0.26465-0. PubMed PMID: 14523103.
43. Hussain M, Becker K, von Eiff C, Peters G, Herrmann M. Analogs of Eap protein are conserved and prevalent in clinical Staphylococcus aureus isolates. Clinical and diagnostic laboratory immunology. 2001; 8(6):1271-6. doi: 10.1128/CDLI.8.6.1271-1276.2001. PubMed PMID: 11687475; PubMed Central PMCID: PMC96261.
44. Geisbrecht B V, Hamaoka B Y, Perman B, Zemla A, Leahy D J. The crystal structures of EAP domains from Staphylococcus aureus reveal an unexpected homology to bacterial superantigens. The Journal of biological chemistry. 2005; 280(17): 17243-50. doi: 10.1074/jbc.M412311200. PubMed PMID: 15691839.
45. Draper S J, Moore A C, Goodman A L, Long C A, Holder A A, Gilbert S C, et al. Effective induction of high-titer antibodies by viral vector vaccines. Nat Med. 2008; 14(8):819-21. doi: 10.1038/nm.1850. PubMed PMID: 18660818.
46. Rollier C S, Reyes-Sandoval A, Cottingham M G, Ewer K, Hill A V. Viral vectors as vaccine platforms: deployment in sight. Curr Opin Immunol. 2011; 23(3):377-82. doi: 10.1016/j.coi.2011.03.006. PubMed PMID: 21514130.

1. A fusion polypeptide comprising:
(i) a first polypeptide, wherein the first polypeptide is an EapH1 polypeptide, or a derivative or variant thereof; and
(ii) a second polypeptide, wherein the second polypeptide is an EapH2 polypeptide, or a derivative or variant thereof.

2. A fusion polypeptide as claimed in claim 1, wherein the first polypeptide:
(a) is a polypeptide which has an amino acid sequence having at least 80%, 85%, 90%, 93%, 95%, 99% or 100% identity to SEQ ID NO: 2 or SEQ ID NO: 4, or
(b) is a fragment of polypeptide (a),
wherein the first polypeptide has the ability to induce antibodies which bind to a Staphylococcus EapH1 polypeptide.

3. A fusion polypeptide as claimed in claim 2, wherein the first polypeptide is a polypeptide which has an amino acid sequence having at least 93% amino acid sequence identity to SEQ ID NO: 4.

4. A fusion polypeptide as claimed in any one of the preceding claims, wherein the second polypeptide:
(c) is a polypeptide which has an amino acid sequence having at least 80%, 85%, 90%, 95%, 97%, 99% or 100% identity to SEQ ID NO: 6 or SEQ ID NO: 8, or
(d) is a fragment of polypeptide (c),
wherein the second polypeptide has the ability to induce antibodies which bind to a Staphylococcus EapH2 polypeptide.

5. A fusion polypeptide as claimed in claim 4, wherein the second polypeptide is a polypeptide which has an amino acid sequence having at least 97% amino acid sequence identity to SEQ ID NO: 8.

6. A fusion polypeptide as claimed in any one of the preceding claims, wherein the fusion polypeptide additionally comprises:
(iii) a third polypeptide, wherein the third polypeptide comprises at least one Eap MAP domain, or a derivative or variant thereof.

7. A fusion polypeptide as claimed in claim 6, wherein the third polypeptide:
(e) is a polypeptide which has an amino acid sequence having at least 80%, 85%, 90% or 95% identity to SEQ ID NO: 9, or
(f) is a fragment of polypeptide (e),
wherein the third polypeptide has the ability to induce antibodies which bind to a Staphylococcus Eap polypeptide.

8. A fusion polypeptide as claimed in any one of the preceding claims, wherein the fusion polypeptide additionally comprises a BitC, EsxA, ClfB, Spa and/or Hla polypeptide.

9. A virus-like particle comprising a fusion polypeptide as claimed in any one of claims 1 to 8.

10. An antibody against a fusion polypeptide as claimed in any one of claims 1 to 8, wherein the antibody:
(a) disrupts or prevents binding of EapH1 to EapH2 or to Eap; or
(b) disrupts or prevents binding of EapH2 to EapH1 or to Eap.

11. A nucleic acid molecule which codes for a fusion polypeptide or a virus-like particle as claimed in any one of claims 1 to 9.

12. A vector or plasmid comprising a nucleic acid molecule as claimed in claim 11, preferably wherein the vector is an adenoviral vector or a Modified Vaccinia Ankara viral vector, most preferably a non-replicating adenoviral vector or a non-replicating Modified Vaccinia Ankara viral vector.

13. A host cell comprising a vector or plasmid as claimed in claim 11, preferably wherein the host cell is a mammalian host cell, more preferably a yeast host cell.

14. A pharmaceutical composition comprising a fusion polypeptide or a virus-like particle as claimed in any one of claims 1 to 9, an antibody as claimed in claim 10, a nucleic acid molecule as claimed in claim 11, or a vector or plasmid as claimed in claim 12, optionally together with one or more pharmaceutically-acceptable carriers, excipients or diluents.

15. A vaccine composition comprising a fusion polypeptide or virus-like particle as claimed in any one of claims 1 to 9, a nucleic acid molecule as claimed in claim 11, or a vector or plasmid as claimed in claim 12, together with a pharmaceutically-acceptable adjuvant.

16. A fusion polypeptide or virus-like particle as claimed in any one of claims 1 to 9, an antibody as claimed in claim 10, a nucleic acid molecule as claimed in claim 11, or a vector or plasmid as claimed in claim 11, for use in therapy or for use as a medicament.

17. A fusion polypeptide or virus-like particle as claimed in any one of claims 1 to 9, an antibody as claimed in claim 10, a nucleic acid as claimed in claim 11, or a vector or plasmid as claimed in claim 11, for use in a method of preventing or treating a Staphylococcus aureus infection in a subject, for use in a method of preventing kidney abscesses due to a Staphylococcus aureus infection in a subject, for use in a method of reducing carriage of Staphylococcus aureus bacteria in a subject or for use in a method of inducing a T-cell response or a B-cell response to a Staphylococcus aureus antigen in a subject.

18. Use of a fusion polypeptide or virus-like particle as claimed in any one of claims 1 to 9, an antibody as claimed in claim 10, a nucleic acid molecule as claimed in claim 11, or a vector or plasmid as claimed in claim 11, in the manufacture of a medicament for use in a method of preventing or treating a Staphylococcus aureus infection in a subject, for use in a method of preventing kidney abscesses due to a Staphylococcus aureus infection in a subject, for use in a method of reducing carriage of Staphylococcus aureus bacteria in a subject, or for use in a method of inducing a T-cell response or a B-cell response to a Staphylococcus aureus antigen in a subject.

19. A method of treating a subject susceptible to Staphylococcus aureus infection comprising administering an effective amount of a fusion polypeptide or virus-like particle as claimed in any one of claims 1 to 9, an antibody as claimed in claim 10, a nucleic acid molecule as claimed in claim 11, a vector or plasmid as claimed in claim 11, or a composition as claimed in claim 14, to the subject.

20. A method of preventing kidney abscesses due to a Staphylococcus aureus infection in a subject comprising administering an effective amount of a fusion polypeptide or virus-like particle as claimed in any one of claims 1 to 9, an antibody as claimed in claim 10, a nucleic acid molecule as claimed in claim 11, a vector or plasmid as claimed in claim 11, or a composition as claimed in claim 14, to the subject.

21. A method of reducing carriage of Staphylococcus aureus bacteria in a subject comprising administering an effective amount of a fusion polypeptide or virus-like particle as claimed in any one of claims 1 to 9, an antibody as claimed in claim 10, a nucleic acid molecule as claimed in claim 11, a vector or plasmid as claimed in claim 11, or a composition as claimed in claim 14, to the subject.

22. A method of inducing a T-cell response or a B-cell response to a Staphylococcus aureus antigen in a subject comprising administering an effective amount of a fusion polypeptide or virus-like particle as claimed in any one of claims 1 to 9, an antibody as claimed in claim 10, a nucleic acid molecule as claimed in claim 11, a vector or plasmid as claimed in claim 11, or a composition as claimed in claim 14, to the subject.

23. A method of preventing or treating a Staphylococcus aureus infection in a subject, of inducing a T-cell response or a B-cell response to a Staphylococcus aureus antigen in a subject, the method comprising the steps of:
(i) administering a first priming amount of a fusion polypeptide or virus-like particle as claimed in any one of claims 1 to 9, an antibody as claimed in claim 10, a nucleic acid molecule as claimed in claim 11, a vector or plasmid as claimed in claim 11, or a composition as claimed in claim 14, to the subject; and then
(ii) administering a second boosting amount of a fusion polypeptide as claimed in any one of claims 1 to 9, an antibody as claimed in claim 10, a nucleic acid molecule as claimed in claim 11, a vector or plasmid as claimed in claim 11, or a composition as claimed in claim 14, to the subject.

24. A method as claimed in claim 23, the method comprising:
(i) administering a vector as claimed in claim 11, wherein the vector is an adenovirus vector (e.g. AdHu5) to the subject; and then
(ii) administering a vector as claimed in claim 11, wherein the vector is a non-replicating poxvirus vector to the subject (e.g. MVA).

25. A process for the production of a fusion polypeptide or virus-like particle as claimed in any one of claims 1 to 9, which process comprises expressing a nucleic acid molecule coding for said fusion polypeptide or virus-like particle in a suitable host and recovering the produced fusion polypeptide or virus-like particle.
